SEQUENCE LISTING (16 sequences)

SEQ ID NO: 1 - DNA, 21 bp, synthetic construct
  source 1..21: mol_type = other DNA
  misc_feature 1..21: note = Exemplary hybridizing region of oligonucleotide probes
cgttcctaccgccctaattat

SEQ ID NO: 2 - DNA, 10 bp, synthetic construct
  source 1..10: mol_type = other DNA
  misc_feature 1..10: note = Exemplary overhang of oligonucleotide probes
ggcttacgcc

SEQ ID NO: 3 - DNA, 10 bp, synthetic construct
  source 1..10: mol_type = other DNA
  misc_feature 1..10: note = Exemplary overhang of oligonucleotide probes
caagaacgta

SEQ ID NO: 4 - DNA, 31 bp, synthetic construct
  source 1..31: mol_type = other DNA
  misc_feature 1..31: note = Exemplary oligonucleotide probe
ggcttacgcccgttcctaccgccctaattat

SEQ ID NO: 5 - DNA, 31 bp, synthetic construct
  source 1..31: mol_type = other DNA
  misc_feature 1..31: note = Exemplary oligonucleotide probe
cgttcctaccgccctaattatcaagaacgta

SEQ ID NO: 6 - DNA, 20 bp, synthetic construct
  source 1..20: mol_type = other DNA
  misc_feature 1..20: note = Exemplary Splint
ggcgtaagcctacgttcttg

SEQ ID NO: 7 - DNA, 41 bp, synthetic construct
  source 1..41: mol_type = other DNA
  misc_feature 1..41: note = Exemplary oligonucleotide probe
ggcttacgcccgttcctaccgccctaattatcaagaacgta

SEQ ID NO: 8 - DNA, 65 bp, synthetic construct
  source 1..65: mol_type = other DNA
  misc_feature 1..65: note = Exemplary oligonucleotide probe
ggcttacgcccgttcctaccgccctaattattttcgttcctaccgccctaattatcaagaacgta

SEQ ID NO: 9 - DNA, 23 bp, synthetic construct
  source 1..23: mol_type = other DNA
  misc_feature 1..23: note = Exemplary oligonucleotide probe
cgttcctacctttcgttcctacc

SEQ ID NO: 10 - DNA, 25 bp, synthetic construct
  source 1..25: mol_type = other DNA
  misc_feature 1..25: note = Exemplary oligonucleotide probe
gccctaattattttgccctaattat

SEQ ID NO: 11 - DNA, 10 bp, synthetic construct
  source 1..10: mol_type = other DNA
  misc_feature 1..10: note = Exemplary hybridizing region
cgttcctacc

SEQ ID NO: 12 - DNA, 11 bp, synthetic construct
  source 1..11: mol_type = other DNA
  misc_feature 1..11: note = Exemplary hybridizing region
gccctaattat

SEQ ID NO: 13 - DNA, 30 bp, synthetic construct
  source 1..30: mol_type = other DNA
  misc_feature 1..30: note = Exemplary oligonucleotide probe
cgttcctacctcaccgcttccgttcctacc

SEQ ID NO: 14 - DNA, 32 bp, synthetic construct
  source 1..32: mol_type = other DNA
  misc_feature 1..32: note = Exemplary oligonucleotide probe
gccctaattatgaagcggtgagccctaattat

SEQ ID NO: 15 - DNA, 10 bp, synthetic construct
  source 1..10: mol_type = other DNA
  misc_feature 1..10: note = Exemplary Linker
tcaccgcttc

SEQ ID NO: 16 - DNA, 10 bp, synthetic construct
  source 1..10: mol_type = other DNA
  misc_feature 1..10: note = Exemplary Linker
gaagcggtga

(19) United States
(12) Patent Application Publication
(10) Pub. No.: US 2023/0323437 A1
(43) Pub. Date: Oct. 12, 2023

(54) METHODS AND COMPOSITIONS FOR STABILIZING AND/OR COMPACTING NUCLEIC ACID

(71) Applicant: 10x Genomics, Inc., Pleasanton, CA (US)

(72) Inventors: Hong CHEN, Fremont, CA (US); Evan R. DAUGHARTHY, Cambridge, MA (US); Shoshoni DROZ, Boston, MA (US); Milena LAZOVA, Medford, MA (US); Zhao ZHAO, Newton, MA (US)

(73) Assignee: 10x Genomics, Inc., Pleasanton, CA (US)

(21) Appl. No.: 17/816,991

(22) Filed: Aug. 2, 2022

Related U.S. Application Data
(60) Provisional application No. 63/228,852, filed on Aug. 3, 2021.

(51) Int. Cl. C12Q 1/6806; C12Q 1/6841

(52) U.S. Cl. CPC C12Q 1/6806; C12Q 1/6841

(57) ABSTRACT

The present disclosure in some aspects relates to methods and compositions for accurately detecting and quantifying multiple analytes present in a biological sample. In some aspects, the methods and compositions provided herein address one or more issues associated with the stability and/or size of nucleic acid structures such as rolling circle amplification products in the biological sample.

### CROSS-REFERENCE TO RELATED APPLICATIONS

This application claims priority to U.S. Provisional Pat. Application No. 63/228,852 filed Aug. 3, 2021, entitled “METHODS AND COMPOSITIONS FOR STABILIZING AND/OR COMPACTING NUCLEIC ACID,” which is herein incorporated by reference in its entirety for all purposes.

### REFERENCE TO AN ELECTRONIC SEQUENCE LISTING

The contents of the electronic sequence listing (202412009700SUBSEQLIST.xml; Size: 21,554 bytes; and Date of Creation: Sep. 14, 2022) is herein incorporated by reference in its entirety.

### FIELD

The present disclosure relates in some aspects to methods and compositions for analyzing a biological sample, such as for stabilizing and/or compacting nucleic acid molecules, structures, and/or complexes using staples in the biological sample for in situ analysis.

### BACKGROUND

Methods are available for analyzing nucleic acids in a biological sample in situ, such as a cell or a tissue. For instance, advances in single molecule fluorescent hybridization (smFISH) have enabled nanoscale-resolution imaging of RNA in cells and tissues. However, oligonucleotide probe-based assay methods for in situ analysis may suffer from low sensitivity, specificity, and/or detection efficiency and may require careful and laborious optimization. Improved methods for in situ analysis are needed. The present disclosure addresses these and other needs.

### BRIEF SUMMARY

There is a need for increasing the stability and/or compaction of structures comprising nucleic acid molecules (e.g., nucleic acid concatemers such as rolling circle amplification products) during in situ analysis of a biological sample. For example, probe/target hybridization complexes and/or rolling circle amplification products may become destabilized (e.g., when wash conditions are too stringent), which may result in a decrease in the number of useful signals, impaired spatial fidelity of signals between hybridization cycles, and loss of information in the assay. Furthermore, compaction of nucleic acid in the sample (e.g., a cell or tissue sample) may allow for better resolution of signals.

Provided herein are methods and compositions for compaction and/or stabilization of structures comprising nucleic acid molecules in situ in a biological sample, including methods for enhancing the stability and/or compaction of nucleic acid molecules during in situ analysis, e.g., for decoding nucleic acid barcode sequences through sequential cycles of detectable probe hybridization (directly or indirectly) to the nucleic acid molecules (e.g., nucleic acid concatemers such as rolling circle amplification products).

In some embodiments, disclosed herein is a method comprising generating a nucleic acid concatemer outside a biological sample. In some embodiments, the generated nucleic acid concatemer can be deposited or attached (e.g., immobilized) to a substrate (e.g., a planar substrate or a bead) or matrix (e.g., a hydrogel matrix). In some embodiments, the generated nucleic acid concatemer comprises multiple copies of an oligonucleotide probe recognition site. In some embodiments, the method further comprises contacting the generated nucleic acid concatemer with one or more oligonucleotide probes, where each oligonucleotide probe comprises (i) a hybridizing region that hybridizes to the oligonucleotide probe recognition site or a portion thereof, and (ii) an overhang that does not hybridize to the nucleic acid concatemer. In some embodiments, the method further comprises connecting the overhangs of the one or more oligonucleotide probes hybridized to different copies of the oligonucleotide probe recognition site, thereby stabilizing and/or compacting the nucleic acid concatemer.

In some embodiments, disclosed herein is a method comprising generating a nucleic acid concatemer in a biological sample. In some embodiments, the method comprises generating a nucleic acid concatemer in situ in the biological sample.

In some embodiments, disclosed herein is a method for analyzing a biological sample, comprising (a) contacting the biological sample with one or more oligonucleotide probes, wherein a nucleic acid concatemer comprising multiple copies of an oligonucleotide probe recognition site is generated in the biological sample, and each oligonucleotide probe comprises (i) a hybridizing region that hybridizes to the oligonucleotide probe recognition site or a portion thereof, and (ii) an overhang that does not hybridize to the nucleic acid concatemer; and (b) connecting the overhangs of the one or more oligonucleotide probes hybridized to different copies of the oligonucleotide probe recognition site.

In some embodiments, the biological sample may be in contact with the one or more oligonucleotide probes prior to or at the initiation of the generation of the nucleic acid concatemer. In some embodiments, the method comprises contacting the biological sample with the one or more oligonucleotide probes prior to or as the generation of the nucleic acid concatemer in the biological sample is initiated. In any of the embodiments herein, the biological sample may be in contact with the one or more oligonucleotide probes during the generation of the nucleic acid concatemer. In some embodiments, the method comprises contacting the biological sample with the one or more oligonucleotide probes after the initiation of the nucleic acid concatemer generation, and/or as the nucleic acid concatemer is being generated in the biological sample. In any of the embodiments herein, the biological sample may be in contact with the one or more oligonucleotide probes after the generation of the nucleic acid concatemer. In some embodiments, the method comprises contacting the biological sample with the one or more oligonucleotide probes after the nucleic acid concatemer is generated, and/or after the nucleic acid concatemer generation is completed. In some embodiments, the method comprises contacting the biological sample containing a nucleic acid concatemer with the one or more oligonucleotide probes, allowing the one or more oligonucleotide probes to bind to the nucleic acid concatemer, and extending the nucleic acid concatemer, e.g., by resuming rolling circle amplification of a circular template that has been used to generate the nucleic acid concatemer. In any of the embodiments herein, the biological sample can be a tissue sample, a sample of isolated and/or cultured cells, an organoid, or a substrate or matrix having nucleic acid molecules (e.g., nucleic acid concatemers) attached thereto. In any of the embodiments herein, the biological sample can be embedded in a matrix (e.g., hydrogel). In some embodiments, the embedded biological sample is cleared, and one or more nucleic acid molecules and/or probes can be attached to the matrix. In some embodiments, one or more products of the nucleic acid molecules and/or probes can be generated in the embedded and cleared biological sample, and the one or more products can be attached to the matrix. For instance, a rolony can be generated in situ from a nucleic acid molecule and/or a probe attached to a hydrogel matrix, and contacted with the one or more oligonucleotide probes that bind to and stabilize and/or compact the rolony. In some embodiments, the rolony can be attached to the hydrogel matrix by crosslinking and/or via functional groups on the one or more oligonucleotide probes.

In any of the embodiments herein, the biological sample is in contact with the one or more oligonucleotide probes after the generation of the nucleic acid concatemer.

In any of the embodiments herein, in the connecting step, the ends of the overhangs may not be hybridized to a splint. In any of the embodiments herein, in the connecting step, the ends of the overhangs may be hybridized to a splint. In some embodiments, the splint may be between about 5 and about 50 nucleotides in length. In some embodiments, the splint may be about 10, about 15, about 20, or about 25 nucleotides in length. In any of the embodiments herein, the splint may be separate from the one or more oligonucleotide probes and the nucleic acid concatemer, and/or the splint does not hybridize to the nucleic acid concatemer. In any of the embodiments herein, the splint may be not covalently labeled with a detectably label, or wherein the splint may be covalently labeled with a detectably label via an optional linker. In any of the embodiments herein, the splint may comprise a sequence that hybridizes to the nucleic acid concatemer.

In any of the embodiments herein, in the connecting step, the overhangs may be not covalently connected. In any of the embodiments herein, in the connecting step, the overhangs may be covalently connected to one another.

In any of the embodiments herein, the connecting step may comprise ligating the ends of the overhangs using enzymatic ligation and/or chemical ligation. In any of the embodiments herein, the connecting step may comprise ligating the ends of the overhangs using template dependent ligation and/or template independent ligation. In any of the embodiments herein, the 5′ and/or 3′ ends of the one or more oligonucleotide probes may comprise click functional groups. In any of the embodiments herein, the click functional groups may selected from the group consisting of: (i) 3′-azido / 5′-alkynyl; (ii) 3′-alkynyl / 5′-azido; (iii) 3′-azido / 5′-cyclooctynyl; (iv) 3′-cyclooctynyl / 5′-azido; (v) 3′-tetrazine / 5′-dienophile; (vi) 3′-dienophile / 5′-tetrazine; (vii) 3′-thiol / 5′-alkynyl; (viii) 3′-alkynyl / 5′-thiol; (ix) 3′-cyano / 5′-1,2-amino thiol; (x) 3′-1,2-amino thiol / 5′-cyano; (xi) 3′-nitrone / 5′-cyclooctynyl; and (xii) 3′-cyclooctynyl / 5′-nitrone. In any of the embodiments herein, the one or more oligonucleotide probes may comprise a 5′ azide and a 3′ alkyne.

In any of the embodiments herein, in the connecting step, the ends of an oligonucleotide probe may be connected, wherein the oligonucleotide probe may comprise (i) hybridizing regions that hybridize to different copies of the oligonucleotide probe recognition site and (ii) a linker between the hybridizing regions that does not hybridize to the nucleic acid concatemer. In some embodiments, the linker is between about 0 and about 200 nucleotides in length. In any of the embodiments herein, the oligonucleotide probe may comprise a 5′ azide and a 3′ alkyne, and a 5′ sequence and a 3′ sequence that hybridize to a splint, and wherein upon hybridization to the splint, the 5′ azide and 3′ alkyne are in proximity for click reaction.

In any of the embodiments herein, in the connecting step, an end of a first oligonucleotide probe and an end of a second oligonucleotide probe may be connected, and wherein the first and second oligonucleotide probes may comprise hybridizing regions that hybridize to different copies of the oligonucleotide probe recognition site. In any of the embodiments herein, the first oligonucleotide probe may comprise a 5′ azide and a 5′ sequence that hybridizes to a splint, the second oligonucleotide probe may comprise a 3′ alkyne and a 3′ sequence that hybridizes to the splint, and upon hybridization of the first and second oligonucleotide probes to the splint, the 5′ azide and 3′ alkyne are in proximity for click reaction.

In any of the embodiments herein, the first oligonucleotide probe may comprise (i) a 5′ azide and a 5′ sequence that hybridizes to a first splint, and (ii) a 3′ alkyne and a 3′ sequence that hybridizes to a second splint, the second oligonucleotide probe may comprise (i) a 3′ alkyne and a 3′ sequence that hybridizes to the first splint, and (ii) a 5′ azide and a 5′ sequence that hybridizes to the second splint, and upon hybridization of the first and second oligonucleotide probes to the first and second splints, the 5′ azide of the first oligonucleotide and the 3′ alkyne of the second oligonucleotide are in proximity for click reaction, and the 3′ alkyne of the first oligonucleotide and the 5′ azide of the second oligonucleotide are in proximity for click reaction.

In any of the embodiments herein, the hybridizing regions of the one or more oligonucleotide probes are independently between about 5 and about 30 nucleotides in length. In some embodiments, the hybridizing regions are independently about 10, about 15, about 20, or about 25 nucleotides in length. In any of the embodiments herein, the hybridizing regions of the one or more oligonucleotide probes may not hybridize to each other.

In any of the embodiments herein, the overhangs of the one or more oligonucleotide probes may be independently between about 3 and about 25 nucleotides in length. In some embodiments, the overhangs may be independently about 6, about 8, about 10, about 12, about 14, or about 16 nucleotides in length. In any of the embodiments herein, the overhangs of the same oligonucleotide probe may not hybridize to each other.

In any of the embodiments herein, the one or more oligonucleotide probes are not covalently labeled with a detectable label. In any of the embodiments herein, one or more of the one or more oligonucleotide probes may be covalently labeled with a detectable label via an optional linker.

In any of the embodiments herein, the oligonucleotide probe recognition site may comprise a first recognition sequence and a second recognition sequence. In any of the embodiments herein, the first and second recognition sequences may be identical or different in sequence. In any of the embodiments herein, the first recognition sequence and the second recognition sequence may be identical or different in length.

In any of the embodiments herein, the nucleic acid concatemer may be generated in situ in the biological sample. In any of the embodiments herein, the nucleic acid concatemer may be immobilized in the biological sample. In any of the embodiments herein, the nucleic acid concatemer may be crosslinked to one or more other molecules in the biological sample.

In any of the embodiments herein, the biological sample may be embedded in a matrix. In some embodiments, the matrix is a hydrogel. In any of the embodiments herein, the nucleic acid concatemer may comprise one or more functional groups for attachment to the matrix. In any of the embodiments herein, the nucleic acid concatemer may be attached to the matrix. In any of the embodiments herein, the one or more oligonucleotide probes may comprise one or more functional groups for attachment to the matrix. In some embodiments, the one or more oligonucleotide probes may comprise a 5′ azide and the matrix comprises a 3′ alkyne. In any of the embodiments herein, the one or more oligonucleotide probes may be attached to the matrix.

In any of the embodiments herein, the nucleic acid concatemer may be hybridized to multiple molecules of the one or more oligonucleotide probes.

In any of the embodiments herein, the nucleic acid concatemer may be detected in situ in the biological sample. In some embodiments, the nucleic acid concatemer is a rolling circle amplification product of a circularizable probe or probe set (e.g., padlock probe) comprising one or more ribonucleotides. In some embodiments, the circularizable probe or probe set (e.g., padlock probe)comprises no more than four consecutive ribonucleotides. In some embodiments, the one or more ribonucleotides are at and/or near a ligatable 3′ end of the padlock probe. In some embodiments, circularizable probe or probe set (e.g., padlock probe)comprises a ribonucleotide at its 3′ end.

In any of the embodiments herein, the nucleic acid concatemer may be detected in situ in the biological sample. In any of the embodiments herein, the method may further comprise imaging the biological sample to detect the nucleic acid concatemer. In some embodiments, the imaging comprises fluorescent microscopy.

In any of the embodiments herein, a sequence of the nucleic acid concatemer molecule may be analyzed in situ in the biological sample. In any of the embodiments herein, the sequence of the nucleic acid concatemer may be analyzed by sequential hybridization, sequencing by hybridization, sequencing by ligation, sequencing by synthesis, sequencing by binding, or a combination thereof. In any of the embodiments herein, the sequence that is analyzed may comprise a barcode sequence. In some embodiments, the barcode sequence corresponds to an analyte or a portion thereof or a labelling agent for an analyte or portion thereof in the biological sample.

In any of the embodiments herein, the nucleic acid concatemer may be a product of an endogenous nucleic acid molecule in the biological sample. In some embodiments, the product is an extension product, a replication product, a reverse transcription product, and/or a rolling circle amplification (RCA) product. In some embodiments, the endogenous nucleic acid molecule may be a viral or cellular DNA or RNA. In some embodiments, the endogenous nucleic acid molecule may be genomic DNA/RNA, mRNA, or cDNA.

In any of the embodiments herein, the nucleic acid concatemer may be a product of a labelling agent that directly or indirectly binds to an analyte in the biological sample. In some embodiments, the product may be an extension product, a replication product, a reverse transcription product, and/or a rolling circle amplification (RCA) product. In any of the embodiments herein, the labelling agent may comprise a reporter oligonucleotide. In some embodiments, the reporter oligonucleotide may comprise one or more barcode sequences and the nucleic acid concatemer comprises one or a plurality of copies of the one or more barcode sequences.

In any of the embodiments herein, the nucleic acid concatemer may be a rolling circle amplification (RCA) product (RCP) of a circular or circularizable probe or probe set that hybridizes to a DNA or RNA molecule in the biological sample. In any of the embodiments herein, the nucleic acid concatemers generated from a plurality of different mRNA and/or cDNA molecules may be analyzed, a barcode sequence in a particular circular or circularizable probe or probe set may uniquely correspond to a particular mRNA or cDNA molecule, and the particular circular or circularizable probe or probe set may further comprise an anchor sequence that is common among circular or circularizable probes or probe sets for a subset of the plurality of different mRNA and/or cDNA molecules.

In any of the embodiments herein, the nucleic acid concatemer may be in the form of a nanoball having a diameter of between about 0.1 µm and about 3 µm. In some embodiments, the diameter is between about 0.1 µm and about 0.5 µm, between about 0.5 µm and about 1 µm, between about 0.8 µm and about 1.3 µm, or between about 1 µm and about 1.5 µm. In any of the embodiments herein, the nucleic acid concatemer may be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length. In any of the embodiments herein, the nucleic acid concatemer may comprise between about 10 and about 100, between about 100 and about 1,000, between about 1,000 and about 5,000, between about 5,000 and about 10,000, or more than 10,000 copies of a unit sequence. In some embodiments, the unit sequence comprises the oligonucleotide probe recognition site and a barcode sequence or complement thereof.

In any of the embodiments herein, the biological sample may be a non-homogenized tissue sample or a tissue section. In any of the embodiments herein, the biological sample may be a processed or cleared biological sample. In any of the embodiments herein, the biological sample may be a tissue slice between about 1 µm and about 50 µm in thickness. In some embodiments, the tissue slice is between about 5 µm and about 35 µm in thickness. In any of the embodiments herein, the biological sample may be a formalin-fixed, paraffin-embedded (FFPE) sample, a frozen tissue sample, or a fresh tissue sample.

In some aspects, provided herein is a method for analyzing a biological sample, comprising: (a) contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; (b) contacting the biological sample with a first oligonucleotide probe and a second oligonucleotide probe each comprising (i) a hybridizing region complementary to the oligonucleotide probe recognition site or a portion thereof, and (ii) an overhang that does not hybridize to the RCP, wherein the hybridizing regions of the first and second oligonucleotide probes are hybridized to different copies of the oligonucleotide probe recognition site; (c) contacting the biological sample with a splint that hybridizes to the overhangs of the first and second oligonucleotide probes; and (d) ligating the overhangs hybridized to the splint, thereby forming a ligated product of the first and second oligonucleotide probes hybridized to different copies of the oligonucleotide probe recognition site in the RCP. In some embodiments, the ligating step may comprise click ligation. In some embodiments, the first and second oligonucleotide probes may each comprise a 5′ azide and a 3′ alkyne, respectively. In any of the embodiments herein, the biological sample may be embedded in a matrix comprising a 3′ alkyne.

In some aspects, provided herein is a method for analyzing a biological sample, comprising: (a) contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; (b) contacting the biological sample with a first oligonucleotide probe and a second oligonucleotide probe each comprising (i) a hybridizing region complementary to the oligonucleotide probe recognition site or a portion thereof, and (ii) a 5′ overhang and a 3′ overhang that do not hybridize to the RCP, wherein the hybridizing regions of the first and second oligonucleotide probes are hybridized to different copies of the oligonucleotide probe recognition site; (c) contacting the biological sample with a first splint that hybridizes to the 5′ overhang of the first oligonucleotide probe and the 3′ overhang of the second oligonucleotide probe, and with a second splint that hybridizes to the 3′ overhang of the first oligonucleotide probe and the 5′ overhang of the second oligonucleotide probe; and (d) ligating the overhangs hybridized to the first splint and the overhangs hybridized to the second splint, thereby forming a ligated product of the first and second oligonucleotide probes hybridized to different copies of the oligonucleotide probe recognition site in the RCP. In some embodiments, the ligating step may comprise click ligation. In some embodiments, the first and second oligonucleotide probes may each comprise a 5′ azide and a 3′ alkyne. In any of the embodiments herein, the biological sample may be embedded in a matrix comprising a 3′ alkyne.

In some aspects, provided herein is a method for analyzing a biological sample, comprising: (a) contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; (b) contacting the biological sample with an oligonucleotide probe comprising (i) a first hybridizing region and a second hybridizing region, each of which is complementary to the oligonucleotide probe recognition site or a portion thereof, (ii) a linker between the first and second hybridizing regions, and (iii) a 5′ overhang and a 3′ overhang, wherein the linker and the overhangs do not hybridize to the RCP, and wherein the first and second hybridizing regions are hybridized to different copies of the oligonucleotide probe recognition site; (c) contacting the biological sample with a splint that hybridizes to the overhangs of the oligonucleotide probe; and (d) ligating the overhangs hybridized to the splint, thereby forming a ligated product of the oligonucleotide probe hybridized to different copies of the oligonucleotide probe recognition site in the RCP. In some embodiments, the ligating step may comprise click ligation. In some embodiments, the oligonucleotide probe may comprise a 5′ azide and a 3′ alkyne. In any of the embodiments herein, the biological sample may be embedded in a matrix comprising a 3′ alkyne.

In any of the embodiments herein, the ligated product may stabilize the RCP hybridized thereto. In any of the embodiments herein, the ligated product may compact the RCP hybridized thereto. In any of the embodiments herein, the method may further comprise crosslinking the ligated product to itself, to one or more other molecules in the biological sample, and/or to a matrix embedding the biological sample. In some embodiments, the crosslinking may reduce the mobility of the RCP in the biological sample and/or in the matrix.

In some aspects, provided herein is a method for analyzing a biological sample, comprising contacting the biological sample with a first oligonucleotide probe and a second oligonucleotide probe, wherein: a nucleic acid concatemer comprising multiple copies of an oligonucleotide probe recognition site is generated in the biological sample, the oligonucleotide probe recognition site comprises a first recognition sequence and a second recognition sequence, the first oligonucleotide probe comprises: (i) a 5′ hybridizing region that hybridizes to the first recognition sequence or portion thereof, (ii) a linker that does not hybridize to the nucleic acid concatemer, and (iii) a 3′ hybridizing region that hybridizes to the first recognition sequence or portion thereof, the second oligonucleotide probe comprises: (i) a 5′ hybridizing region that hybridizes to the second recognition sequence or portion thereof, (ii) a linker that does not hybridize to the nucleic acid concatemer, and (iii) a 3′ hybridizing region that hybridizes to the second recognition sequence or portion thereof, and the first and second oligonucleotide probes are each hybridized to different copies of the oligonucleotide probe recognition site. In some embodiments, the linkers of the first and second oligonucleotide probes may be not hybridized to each other. In any of the embodiments herein, the linkers of the first and second oligonucleotide probes may be hybridized to each other.

In some embodiments, disclosed herein is a kit comprising an oligonucleotide probe and a splint, wherein: the oligonucleotide probe comprises (i) a hybridizing region that hybridizes to an oligonucleotide probe recognition site or portion thereof on a nucleic acid concatemer, and (ii) an overhang that does not hybridize to the nucleic acid concatemer, and the splint comprises a sequence that hybridizes to the overhang or a portion thereof of the oligonucleotide probe. The kit can be used in a method of any of the embodiments disclosed herein.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary oligonucleotide probe set hybridized to one or more nucleic acid concatemers. The hybridizing region of a first oligonucleotide probe is hybridized to a first oligonucleotide probe recognition site in a nucleic acid concatemer, which recognition site is complementary to the hybridizing region in the first oligonucleotide probe. The hybridizing region of a second oligonucleotide probe is hybridized to a second oligonucleotide probe recognition site in the same or a different nucleic acid concatemer, which recognition site is complementary to the hybridizing region in the second oligonucleotide probe. The first and second oligonucleotide probe recognition sites can be in the same nucleic acid concatemer or in different nucleic acid concatemers. The overhangs of the first and second oligonucleotide probes can be connected, e.g., via enzymatic ligation between a 5′ end (e.g., 5′-phosphate) and a 3′ end (e.g., 3′-hydroxyl), or via chemical ligation of the functional groups present at the ends of the overhangs. Exemplary functional groups include click functional groups, such as alkyne and azide in the oligonucleotide probes (e.g., 5′ azide in the first oligonucleotide probe and 3′ alkyne in the second oligonucleotide probe). Optionally a splint (e.g., a “staple”) can be used to facilitate connection of the overhangs. The splint can hybridize to a first splint recognition region in the overhang of the first oligonucleotide probe, and to a second splint recognition region in the overhang of the second oligonucleotide probe. The splint may optionally comprise a region (indicated by dashed line in the splint) that hybridizes to the one or more nucleic acid concatemers.

FIG. 2 shows an exemplary oligonucleotide probe hybridized to a nucleic acid concatemer at the oligonucleotide probe recognition site via the hybridizing region of the oligonucleotide probe. A functional group at the terminus of the overhang of the oligonucleotide probe (e.g., a portion of the oligonucleotide probe that does not hybridize to the nucleic acid concatemer) can be reacted with another functional group in a matrix (e.g., a hydrogel matrix) in which the sample containing the nucleic acid concatemer is embedded. Exemplary functional groups include click functional groups, such as alkyne in the matrix and azide in the oligonucleotide probe (e.g., 5′ azide in the oligonucleotide probe).

FIG. 3 illustrates an exemplary oligonucleotide probe design. A first oligonucleotide probe comprises a hybridizing region complementary to a first oligonucleotide probe recognition site in a nucleic acid concatemer, and the hybridizing region is flanked by two overhangs that do not hybridize to the nucleic acid concatemer. A second oligonucleotide probe comprises a hybridizing region complementary to a second oligonucleotide probe recognition site in the same or a different nucleic acid concatemer, and the hybridizing region is flanked by two overhangs that do not hybridize to the same or different nucleic acid concatemer. The ends of the overhangs of the first oligonucleotide probe may be connected to the ends of the overhangs of the second oligonucleotide probe. The overhangs of the first and second oligonucleotide probes can each be connected via enzymatic ligation between a 5′ end (e.g., 5′-phosphate) and a 3′ end (e.g., 3′-hydroxyl), or via chemical ligation of the functional groups present at the ends of the overhangs. The two connections can be independent of one another, e.g., one via enzymatic ligation and the other via chemical ligation. Exemplary functional groups include click functional groups, such as alkyne and azide in the oligonucleotide probes (e.g., 5′ azide in one oligonucleotide probe and 3′ alkyne in the other oligonucleotide probe). The two connections can be independently templated ligation or non-templated ligation. For instance, a splint can be used to facilitate ligation of the ends of the overhangs, and a splint can hybridize to a first splint recognition region in one overhang and to a second splint recognition region in another overhang. The first and second oligonucleotide probes can be the same or different. Further, the hybridizing regions of the first and second oligonucleotide probes can have the same sequence or different sequences.

FIG. 4 shows an exemplary oligonucleotide probe set hybridized to a nucleic acid concatemer. A first oligonucleotide probe comprises a first hybridizing region hybridized to a first copy of an oligonucleotide probe recognition site on the nucleic acid concatemer. The first copy of the oligonucleotide probe recognition site can be complementary to the first hybridizing region. A second oligonucleotide probe can mediate the connection between one molecule of the first oligonucleotide probe and another molecule of the first oligonucleotide probe. A functional group at the terminus of an overhang of the first oligonucleotide probe can be reacted with a functional group in another molecule of the first oligonucleotide probe, with a functional group in another molecule of the second oligonucleotide probe, or with a functional group in a matrix (e.g., a hydrogel matrix) in which the sample containing the nucleic acid concatemer is embedded. In cases where the overhang of the first oligonucleotide probe is connected to another molecule of the second oligonucleotide probe (not shown), the connection can be facilitated (e.g., templated) by a splint, and the free functional group of the another molecule of the second oligonucleotide probe can be reacted with a functional group in the matrix. Exemplary functional groups include click functional groups, such as alkyne in the matrix and azide in the oligonucleotide probe (e.g., 5′ azide in the first oligonucleotide probe that has not reacted with 3′ alkyne in the second oligonucleotide probe). Either end of the oligonucleotide probes can be reacted with the hydrogel matrix, e.g., depending on the class of functional group(s) present at the termini of the oligonucleotide probes. The first and second oligonucleotide probes can be the same or different. Further, the hybridizing regions of the first and second oligonucleotide probes can have the same sequence or different sequences.

FIG. 5 shows an exemplary oligonucleotide probe design. The oligonucleotide probe comprises, from one end to another: a first overhang (e.g., comprising a first splint recognition region), a first hybridizing region, a second hybridizing region, and a second overhang (e.g., comprising a second splint recognition region). The oligonucleotide probe optionally comprises a linker region between the first and second hybridizing regions that does not hybridize to the nucleic acid concatemer. In some embodiments, the linker region and the first and second overhangs do not hybridize to the nucleic acid concatemer, whereas the first and second hybridizing regions are capable of hybridizing to a first and a second oligonucleotide probe recognition sites, respectively, on the nucleic acid concatemer. The first and second oligonucleotide probe recognition sites can have the same sequence or different sequences. The end of the first overhang has a functional group that may be ligated to a functional group on the end of the second overhang, wherein the ligation may be a splint mediated ligation.

FIG. 6 shows an exemplary oligonucleotide probe set hybridized to a nucleic acid concatemer and a matrix. The first oligonucleotide probe comprises a first hybridizing region that is hybridized to the oligonucleotide probe recognition site of the nucleic acid concatemer. The first oligonucleotide probe further comprises a second hybridizing region that is not hybridized to the nucleic acid concatemer. An overhang of the first oligonucleotide probe can be ligated to a first overhang of a second oligonucleotide probe through the functional groups present at their respective termini. Optionally, the ligation is mediated by a splint that is hybridized to the overhangs (e.g., splint recognition regions) of the first and second oligonucleotide probes. The second oligonucleotide probe can comprise a first hybridizing region hybridized to another copy of the oligonucleotide probe recognition site on the nucleic acid concatemer, and further comprise a second hybridizing region that is not hybridized to the nucleic acid concatemer. An overhang adjacent to the second hybridizing region can be connected to a matrix (e.g., hydrogel) through the functional group at its terminus.

FIG. 7A shows exemplary oligonucleotide probe sets hybridized to the nucleic acid concatemer. The oligonucleotide probe recognition sites on the nucleic acid concatemer comprise a first oligonucleotide probe recognition site immediately adjacent to a second oligonucleotide probe recognition site. A first oligonucleotide probe is hybridized to the nucleic acid concatemer with the first hybridizing region of the probe hybridizing to the first oligonucleotide probe recognition site of the first copy of the oligonucleotide probe recognition site of the nucleic acid concatemer, and the second hybridizing region of the probe hybridizing to the first oligonucleotide probe recognition site of the second copy of the oligonucleotide probe recognition site of the nucleic acid concatemer. A second oligonucleotide probe is hybridized to the nucleic acid concatemer with first hybridizing region of the probe hybridizing to the second oligonucleotide probe recognition site of the second copy of the oligonucleotide probe recognition site of the nucleic acid concatemer, and the second hybridizing region of the probe hybridizing to the second oligonucleotide probe recognition site of the first copy of the oligonucleotide probe recognition site of the nucleic acid concatemer. The probes are hybridized to the nucleic acid concatemer in an orientation that has the termini of the second probe adjacent to the linker region of the first probe (FIG. 7A, upper panel). In other aspects, the hybridizing regions of the oligonucleotide probes can hybridize to adjacent oligonucleotide probe recognition sites, while the other hybridizing regions of the oligonucleotide probes hybridize to non-adjacent oligonucleotide probe recognition sites in the same nucleic acid concatemer or different nucleic acid concatemers (FIG. 7A, lower panel).

FIG. 7B shows an exemplary oligonucleotide probe set hybridized to the nucleic acid concatemer in an alternative configuration. The oligonucleotide probe recognition sites on the nucleic acid concatemer comprise a first oligonucleotide probe recognition site immediately adjacent to a second oligonucleotide probe recognition site. A first oligonucleotide probe is hybridized to the nucleic acid concatemer with the first hybridizing region of the probe hybridizing to the first oligonucleotide probe recognition site of the first copy of the oligonucleotide probe recognition site of the nucleic acid concatemer, and the second hybridizing region of the probe hybridizing to the first oligonucleotide probe recognition site of the second copy of the oligonucleotide probe recognition site of the nucleic acid concatemer. A second oligonucleotide probe is hybridized to the nucleic acid concatemer with first hybridizing region of the probe hybridizing to the second oligonucleotide probe recognition site of the second copy of the oligonucleotide probe recognition site of the nucleic acid concatemer, and the second hybridizing region of the probe hybridizing to the second oligonucleotide probe recognition site of the first copy of the oligonucleotide probe recognition site of the nucleic acid concatemer. The probes are hybridized to the nucleic acid concatemer in an orientation that has the linker region of the second probe adjacent to the linker region of the first probe. All or a portion of the linker sequences of the oligonucleotide probes may be complementary. The linker sequences may hybridize to each other to enhance stability.

FIG. 8A and FIG. 8B illustrate two possible binding configurations of an oligonucleotide probe comprising two hybridizing regions separated by a linker region, and lacking any overhangs (e.g., splint recognition regions). FIG. 8A shows a first possible configuration, wherein one hybridizing region of the oligonucleotide probe is hybridized to an oligonucleotide probe recognition site on the nucleic acid concatemer, while the second hybridizing region remains unhybridized. As the second hybridizing region is not hybridized to the nucleic acid concatemer, it can be detected by a fluorescently labeled probe hybridized to the oligonucleotide probe (“fluorescent signal on probe”). FIG. 8B shows a second possible configuration, wherein both hybridizing regions of an oligonucleotide probe are hybridized to different copies of the oligonucleotide probe recognition site on the nucleic acid concatemer. As both hybridizing regions are hybridized to the nucleic acid concatemer, neither hybridizing region can hybridize to the fluorescently labeled probe. A fluorescently labeled probe that hybridizes to the nucleic acid concatemer to detect the concatemer itself (“fluorescent signal on RCP”) serves as a control.

FIG. 8C illustrates the fluorescent signal on RCP versus fluorescent signal on probe of the two probe hybridization configurations exemplified in FIG. 8A and FIG. 8B. The steep slope of the line of best fit indicates that the fluorescent probe is able to hybridize to hybridizing regions (“free arms”) of the oligonucleotide probes readily, indicating that the oligonucleotide probe of the design presented in FIG. 8A and FIG. 8B frequently only has one hybridizing region of its two hybridizing regions hybridized to the nucleic acid concatemer.

FIG. 8D and FIG. 8E illustrate two possible binding configurations of an oligonucleotide probe comprising two hybridizing regions separated by a linker, and further comprising and two overhangs (e.g., splint recognition regions) on either end. FIG. 8D shows a first possible configuration, wherein one hybridizing region of the oligonucleotide probe is hybridized to an oligonucleotide probe recognition site on the nucleic acid concatemer, while the second hybridizing region remains unhybridized. As the second hybridizing region is not hybridized to the nucleic acid concatemer, it can be detected by a fluorescently labeled probe hybridized to the oligonucleotide probe (“fluorescent signal on probe”). The 5′ and 3′ ends of the oligonucleotide probe have not been connected. FIG. 8E shows a second possible configuration, wherein both hybridizing regions are each hybridized to a different copy of the oligonucleotide probe recognition site on the nucleic acid concatemer. The terminal regions of the splint recognition sites have been connected (e.g., a click ligation reaction) mediated by a splint. As both hybridizing regions are hybridized to the nucleic acid concatemer, neither hybridizing region can hybridize to the fluorescent probe to generate “fluorescent signal on probe.” A fluorescently labeled probe that hybridizes to the nucleic acid concatemer to detect the concatemer itself (“fluorescent signal on RCP”) serves as a control.

FIG. 8F illustrates the fluorescent signal on RCP versus fluorescent signal on probe of the two probe hybridization configurations exemplified in FIG. 8D and FIG. 8E. The slope of the line of best fit significantly less step than that of FIG. 8C, indicating that fewer fluorescent probes are hybridizing to the hybridizing regions of the oligonucleotide probes compared to FIG. 8A and FIG. 8B, as both hybridizing regions of the oligonucleotide probe are more often hybridizing to the nucleic acid concatemer.

FIG. 8G and FIG. 8H illustrate two possible binding configurations of a probe set comprising two oligonucleotide probes, each comprising one hybridizing region flanked on either end with overhangs (e.g., splint recognition regions). FIG. 8G shows a first possible configuration of a pair of oligonucleotide probes with 5′ and 3′ regions ligated together. Optionally, two splints are hybridized with the overhangs of the oligonucleotide probes to mediate the probe ligation. A hybridizing region of one of the connected oligonucleotide probes is hybridized to the oligonucleotide probe recognition site, whereas the hybridizing region of the other oligonucleotide probe is not hybridized to the nucleic acid concatemer, and is free to hybridize to the fluorescent detection probe, resulting in “fluorescent signal on probe” (fluorescent signal on free hybridizing region that is not hybridized to the concatemer). FIG. 8H shows a first possible configuration of a pair of oligonucleotide probes with 5′ and 3′ regions ligated together. Optionally, two splints are hybridized with the overhangs of the oligonucleotide probes to mediate the probe ligation. The individual hybridizing regions of the oligonucleotide probes are hybridized to different copies of the oligonucleotide probe recognition site. As both hybridizing regions are hybridized to the nucleic acid concatemer, neither hybridizing region can hybridize to the fluorescent detection probe to generate “fluorescence on the probe.” A fluorescently labeled probe that hybridizes to the nucleic acid concatemer to detect the concatemer itself (“fluorescent signal on RCP”) serves as a control.

FIG. 8I illustrates the fluorescent signal on RCP versus fluorescent signal on probe of the two probe hybridization configurations exemplified in FIG. 8G and FIG. 8H. The slope of the line of best fit is not as steep as that seen in FIG. 8C, indicating that fewer fluorescent detection probes are hybridizing to the hybridizing regions of the oligonucleotide probes, because both hybridizing regions of the connected oligonucleotide probe set are more often hybridizing the nucleic acid concatemer. The slope is similar to that seen in FIG. 8F, suggesting that the hybridization frequency for these two oligonucleotide probe designs are similar, and both higher than that of the oligonucleotide probe design in FIG. 8A and FIG. 8B.

FIG. 9 shows an exemplary schematic using an oligonucleotide probe set to stabilize and/or compact a nucleic acid concatemer. The nucleic acid concatemer comprises multiple copies of an oligonucleotide probe recognition site. Once the concatemer is generated (e.g., by rolling circle amplification), it is contacted with oligonucleotide probes. The oligonucleotide probes comprise at least one hybridizing region and an overhang region that does not hybridize to the nucleic acid concatemer, hybridize to the oligonucleotide probe recognition sites on the nucleic acid concatemer via hybridizing regions in the oligonucleotide probes. The oligonucleotide probes are then connected to one another via functional groups (e.g., click functional groups) present at the respective 5′ and 3′ ends through a connecting step to generate ligated probes. Optionally, splints are introduced to the concatemer-oligonucleotide probe complexes and hybridize to the overhangs (e.g., at splint recognition regions). These methods stabilize the conformation of the nucleic acid concatemer in the sample, allowing the nucleic acid concatemer to remain in the same position and conformation throughout the concatemer analysis or processing steps. In some cases, the oligonucleotide probe complexes can assist in compacting the concatemer as well as maintaining a stable conformation throughout the analysis process.

FIG. 10 shows exemplary fluorescent images of RCPs at the start and at the end of a sequencing run, comparing the single-oligonucleotide staple approach and a modular splint-guided staple approach disclosed herein. More nucleic acid concatemers remained stable at the end of the sequencing run using the modular splint-guided staple approach.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ sequencing amplicons, such as polymerase colonies (“polonies”) and rolling circle polymerase colonies (“rolonies”), comprise a clonally amplified population of spatially co-localized sequencing templates in situ, such as within a 3D matrix, within a cell, or within a tissue. Upon generation, rolonies are understood to be long unstructured ssDNA molecules containing many tandem repeats of the circular template sequence, which can form DNA nanoballs (“DNB”) under certain conditions, such as in situ rolling circle amplification (RCA). DNBs are not intrinsically stable to environmental changes, such as changes in the local matrix environment such as those that occur during sequencing or sequence detection, including changes in solvent environment, temperature, or exposure to denaturing conditions; nor to temporal processes, such as diffusion and entropic processes, that may cause the DNB to become less compact or localized, e.g., stretched out or diffuse, reducing the signal intensity by virtue of causing the fluorescence signal of the amplicon during detection to be distributed within a larger volume; or upon fragmentation of the DNA backbone, which may occur naturally or be catalyzed by chemical or biochemical reactions or side-reactions, to cause the DNB to “fall apart” wherein fragments are washed out of the sample, reducing the number of tandem copies present for fluorescent detection and thereby reducing the signal from fluorescence detection.

In some aspects, an ideal nucleic acid concatemer (e.g., DNB) for detection in situ, e.g., by in situ sequencing such as fluorescent in situ sequencing (FISSEQ), has the properties of maintaining or substantially maintaining a stable localized nanoball conformation, such as being constrained or substantially constrained to a given 3D volume within the sample; and maintaining or substantially maintaining the original number of tandem repeat copies generated by amplification.

Provided herein are methods involving the use of probes for stabilizing and/or compacting nucleic acid concatemers (e.g., rolling circle amplification (RCA) products (RCP)) or other amplicons such as in situ PCR amplicons (“polonies”). When utilizing RCA to detect nucleic acid targets, properly resolving densely packed RCPs in a cell or surface depends upon resolution of the individual RCPs. RCPs are concatemers comprising multiple copies of a target sequence (or complement thereof) and other probe sequences (e.g., barcode sequences), which can be visualized in situ using detectable probes (e.g., detectably labelled probes that hybridize to RCPs directly or indirectly via intermediate probes). In some embodiments, RCP detection is enhanced by stabilization of the concatemers, e.g., via probes that mediate intra-molecule interaction and/or attachment to one or more other molecules and/or a matrix. In some embodiments, RCP detection is enhanced by compaction of the concatemers, as reduction in size results in local concentration of the detection probes to increase signal intensity. Stabilization and/or compaction also allows for increased resolution of individual RCPs. Stabilization/compaction probes are designed to hybridize to regions of the RCP and pull the concatemer together to reduce its overall size. Oligonucleotide probes such as those described in Clausson et al., Scientific Reports 5: 12317 (2015), the entire contents of which are incorporated herein by reference, depend only on hybridization with the RCP to maintain compaction. Linear stabilization/compaction probes are typically stripped away along with detection probes following rounds of in situ hybridization and target detection. Thus, there is a need for methods and compositions for persistent stabilization and/or compaction of RCPs that are resistant to stripping and/or remain stable in sequential hybridization cycles.

Provided herein are methods involving the use of oligonucleotide probes for compacting and/or stabilizing nucleic acid concatemers (e.g., rolling circle amplification (RCA) products (RCP)). In some aspects, the present disclosure provides methods and compositions for persistent stabilization of RCPs. In some embodiments, circularizable oligonucleotide probes and methods of hybridization and ligation reactions are provided. In some embodiments, the composition and methods disclosed herein allow for stabilization and/or compaction of RCPs generated in the biological sample, comprising multiple copies of an oligonucleotide probe recognition site. In some aspects, provided herein are methods and compositions for stabilizing and/or compacting RCPs using one or more oligonucleotide probes, wherein each oligonucleotide probe comprises a hybridizing region complementary to the oligonucleotide probe recognition site, or a portion thereof, and an overhang that does not hybridize to the RCP. The overhang may be connected to one or more molecules and/or a matrix to stabilize and/or compact the RCP.

In some aspects, provided herein are methods and compositions of stabilizing and/or compacting a nucleic acid concatemer in a biological sample, wherein the nucleic acid concatemer comprises multiple copies of an oligonucleotide probe recognition site and the biological sample is contacted with a plurality of oligonucleotide probes that hybridize to an oligonucleotide probe recognition site on the nucleic acid concatemer. In some embodiments, the nucleic acid concatemer comprises at least two copies of the oligonucleotide probe recognition site, and at least one molecule of the oligonucleotide probe forms a hybridization complex with the recognition sites of the nucleic acid concatemer. In some embodiments, the hybridization complex comprises multiple molecules of the oligonucleotide probe.

In some embodiments, the oligonucleotide probes are introduced during the amplification step. In some embodiments, the oligonucleotide probes are introduced to the RCP after the amplification process. Upon introduction of the oligonucleotide probes either during or after the amplification process, in some embodiments, the oligonucleotide probes hybridize to the oligonucleotide recognition sites on the RCP. In some embodiments, the ends of one or more oligonucleotide probes are connected. In some embodiments, the connecting step comprises ligating the ends of the oligonucleotide probes together to create circularized oligonucleotide probes. In some embodiments, the connecting step comprises ligating the ends of the same oligonucleotide probe hybridized to different copies of the oligonucleotide probe recognition site, by ligating the 5′ end of the oligonucleotide probe to the 3′ end of the same oligonucleotide probe. In some embodiments, the connecting step comprises ligating the ends of two oligonucleotide probes hybridized to different copies of the oligonucleotide probe recognition site by ligating the 5′ end of a first oligonucleotide probe to the 3′ end of a second oligonucleotide probe. In some embodiments, the ends of the oligonucleotide probe are in one of the two hybridizing regions. In some embodiments, the ends of the oligonucleotide probes are in the linker. In some embodiments, the ends of the oligonucleotide probe are in the nonhybridizing (to the nucleic acid concatemer) stem region of the step-loop structure. In some embodiments, creation of the circularized oligonucleotide probe hybridized to the RCP results in compaction and/or stabilization of the RCP. In some embodiments, the stabilized and/or compacted RCP is resistant to stripping treatment and remains stable and/or compacted during repeated cycles of detectable probe hybridization and stripping. In some embodiments, the stabilized and/or compacted RCP can be crosslinked, e.g., by crosslinking the circularized oligonucleotide probe to itself, to one or more other molecules (e.g., the RCP) in the biological sample, and/or to a matrix embedding the biological sample, where the crosslinking reduces the mobility of the RCP in the biological sample.

In some embodiments, disclosed herein is a method comprising a first step in which a first site of an oligonucleotide probe binds a first site of a nucleic acid concatemer, and a second step in which a second site of the oligonucleotide probe binds a second site of the nucleic acid concatemer, thereby forming the intramolecular linkage. In some embodiments, the first step substantially proceeds to completion before the second step has substantially proceeded. Therefore, in some embodiments, the oligonucleotide probe is likely binding the nucleic acid concatemer at only a single site per oligonucleotide probe, rather than at both sites. In some aspects, the benefits to concatemer size and shape are derived from the difference in physical properties of the concatemer in a partially duplexed state, versus a substantially single-stranded state. For example, dsDNA has a longer persistence length (“stiffer”) than ssDNA, and diffuses less rapidly through a porous matrix.

In some embodiments, the RCA reaction efficiency is affected by oligonucleotide probe (e.g., stabilization and/or compaction probe) concentration during RCA. In some embodiments, at high concentrations, the oligonucleotide probe inhibits RCA, such as by competing for Phi29 polymerase binding and/or extension. In some embodiments, at low concentrations, wherein the RCA demonstrates benefits from inclusion of oligonucleotide probes, the oligonucleotide probes may not be abundant enough to occupy a substantial number of sites within each concatemer across a population of concatemers. For example, the final number of oligonucleotide probe binding sites across a population of cpncatemers may be substantially higher than the total number of oligonucleotide probes present in the reaction, causing each concatemer to be occupied at only a subset of binding sites. Alternatively, the oligonucleotide probe may be consumed during RCA, which proceeds in parallel to diffusion of the reactive molecules through the 3D matrix, such that the oligonucleotide probe is substantially consumed during RCA of concatemers proximal to the outer boundary of the matrix, and is therefore substantially consumed by the time the reactive components have diffused into the regions of the matrix distal to the matrix-liquid interface of the reaction.

In some embodiments, disclosed herein is a method of forming intramolecular linkages within nucleic acid concatemers within a 3D matrix with the following properties: efficiently form intramolecular linkages within the concatemer by generation of a final construct having two separate but chemically linked nucleic acid domains hybridized to the concatemer; and occupy a uniform and controllable fraction of sites within concatemers regardless of concatemer position within the 3D matrix.

The present disclosure is applicable to other forms of in situ nucleic acid amplification other than RCA, including, but not limited to, in situ PCR (“polonies”).

### II. Samples, Analytes, and Target Sequences

A method disclosed herein may be used to process and/or analyze any analyte(s) of interest, for example, for detecting the analyte(s) in situ in a sample of interest. A target nucleic acid sequence as disclosed herein may be or be comprised in an analyte (e.g., a nucleic acid analyte, such as genomic DNA, mRNA transcript, or cDNA, or a product thereof, e.g., an extension or amplification product, such as an RCA product) and/or may be or be comprised in a labelling agent for one or more analytes (e.g., a nucleic acid analyte or a non-nucleic acid analyte) in a sample or a product of the labelling agent. Exemplary analytes and labelling agents are described below.

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) and/or products thereof (e.g., amplicons such as RCPs) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (I) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 µm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 µm. Thicker sections can also be used if desired or convenient, e.g., at least 70, 80, 90, or 100 µm or more. Typically, the thickness of a tissue section is between 1-100 µm, 1-50 µm, 1-30 µm, 1-25 µm, 1-20 µm, 1-15 µm, 1-10 µm, 2-8 µm, 3-7 µm, or 4-6 µm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than -15° C., less than -20° C., or less than -25° C.

### (iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circularizable probe or probe set. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., padlock probe).

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (IV) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample comprises cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 µm to about 2 mm.

Additional methods and aspects of hydrogel embedding of biological samples are described for example in Chen et al., Science 347(6221):543-548, 2015, the entire contents of which are incorporated herein by reference.

### (V) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include, but is not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson’s trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright’s stain, Jenner’s stain, Can-Grunwald stain, Leishman stain, and Giemsa stain.

In some embodiments, biological samples can be destained. Any method of destaining or discoloring a biological sample can be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the entire contects of which are incorporated herein by reference.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2x, 2.1x, 2.2x, 2.3x, 2.4x, 2.5x, 2.6x, 2.7x, 2.8x, 2.9x, 3x, 3.1x, 3.2x, 3.3x, 3.4x, 3.5x, 3.6x, 3.7x, 3.8x, 3.9x, 4x, 4.1x, 4.2x, 4.3x, 4.4x, 4.5x, 4.6x, 4.7x, 4.8x, or 4.9x its non-expanded size. In some embodiments, the sample is isometrically expanded to at least 2x and less than 20x of its non-expanded size.

### (VII) Crosslinking and De-Crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Pat. Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods can be used and include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

### B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (e.g. a padlock or other circularizable probe for a nucleic acid analyte) and may lead directly to the generation of a circularized template for RCA. Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being detectable.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### (iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

### (a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a circularizable probe or probe set (e.g., padlock probe, a gapped padlock probe), a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9°N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g, separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe or probe set, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling, for example, by Mu polymerase, DNA polymerase, RNA polymerase, reverse transcriptase, VENT polymerase, Taq polymerase, and/or any combinations, derivatives, and variants (e.g., engineered mutants) thereof. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, a product is a primer extension product of an analyte, a labelling agent, a probe or probe set bound to the analyte (e.g., a circularizable probe bound to genomic DNA, mRNA, or cDNA), or a probe or probe set bound to the labelling agent (e.g., a circularizable probe bound to one or more reporter oligonucleotides from the same or different labelling agents).

In some embodiments, a primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some embodiments, two nucleic acid sequences can become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) such as linear RCA, a branched RCA, a dendritic RCA, or any combination thereof can be used. (See, e.g., Baner et al, Nucleic Acids Research, 26:5073-5078, 1998; Lizardi et al, Nature Genetics 19:226, 1998; Mohsen et al., Acc Chem Res. 2016 November 15; 49(11): 2540-2550; Schweitzer et al. Proc. Natl Acad. Sci. USA 97:101 13- 1 19, 2000; Faruqi et al, BMC Genomics 2:4, 2000; Nallur et al, Nucl. Acids Res. 29:el 18, 2001; Dean et al. Genome Res. 1 1 :1095- 1099, 2001; Schweitzer et al, Nature Biotech. 20:359-365, 2002; U.S. Pat. Nos. 6,054,274, 6,291,187, 6,323,009, 6,344,329 and 6,368,801), the entire contents of each of which are incorporated herein by reference. Exemplary polymerases for use in RCA comprise DNA polymerase such phi29 (cp29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N- hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a probe comprising the hybridizing region comprising one or more photoreactive nucleotides) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe. The exogenously added nucleic acid probe may be optionally ligated to a cellular nucleic acid molecule or another exogenous nucleic acid molecule. In other examples, a product comprising a target sequence for a probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein may a probe hybridizing to an RCP. The probe may comprise an overhang that does not hybridize to the RCP but hybridizes to another probe. The probe may be optionally ligated to a cellular nucleic acid molecule or another probe, e.g., an anchor probe that hybridize to the RCP.

### C. Target Sequences

A target sequence for a probe disclosed herein may be comprised in any analyte disclose herein, including an endogenous analyte (e.g., a viral or cellular nucleic acid), a labelling agent, or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the embodiments herein, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the embodiments herein, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 and US 2021/0164039, which are hereby incorporated by reference in their entirety.

### III. Nucleic Acid Concatemer and RCA

In some aspects, provided herein are methods and compositions for processing a biological sample comprising a nucleic acid concatemer, such as a product (e.g., an amplification product) of an analyte in the biological sample. In some embodiments, the nucleic acid concatemer is a product of a labelling agent that directly or indirectly binds to an analyte in the biological sample. In some embodiments, the analyte is a nucleic acid analyte or a non-nucleic acid analyte, and wherein the labelling agent comprises a reporter oligonucleotide and an analyte-binding moiety coupled thereto.

In some embodiments, the nucleic acid concatemer comprises nucleic acids. In some embodiments, the nucleic acid concatemer contains natural and unnatural nucleotides. For example, the nucleic acid concatemer may comprise modified nucleotides, non-nucleotides, or synthetic nucleotides. Nucleotides amendable to the present application include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. In some embodiments, the modified nucleotides are amine-modified nucleotides.

In some embodiments, the nucleic acid concatemer is between about 1 and about 85 kilobases in length, such as between any of about 1 and about 15 kilobases, about 10 and about 30 kilobases, about 20 and about 40 kilobases, about 30 and about 50 kilobases, about 40 and about 60 kilobases, about 50 and about 70 kilobases, and about 60 and about 85 kilobases in length. In some embodiments, the nucleic acid concatemer is at least 1 kilobase in length, such as any of about 15, 25, 35, 45, 55, 65, or 85 kilobases in length. In some embodiments, the nucleic acid concatemer is more than 85 kilobases in length.

In some embodiments, the nucleic acid concatemer is a rolling circle amplification product comprising a barcode sequence corresponding to an analyte in the biological sample, and the nucleic acid concatemer is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.

In some embodiments, the nucleic acid concatemer forms a nanoball such as one having a diameter between about 0.1 µm and about 3 µm. The nanoball may be a product of amplification, such as RCA. In some embodiments, the nanoball has a diameter of between about 0.1 µm and about 4 µm, such as between any of about 0.1 µm and about 0.5 µm, about 0.2 µm and about 2 µm, about 1 µm and about 3 µm, and about 2 µm and about 4 µm. In some embodiments, the nanoball diameter is at least about 0.1 µm, such as at least any of about 0.2 µm, 0.5 µm, 1 µm, 1.5 µm, 2 µm, 2.5 µm, 3 µm, 3.5 µm, and 4 µm.

In some embodiments, a nucleic acid concatemer is generated from a nucleic acid analyte. For example, the nucleic acid analyte may be DNA, ssDNA, or RNA. In some embodiments, the analyte is a non-nucleic acid analyte (e.g., a protein). In some embodiments, the nucleic acid concatemer is generated from a labelling agent that directly or indirectly binds to an analyte in the biological sample. Examples of labelling agents are described in Section II.B.ii.

In some aspects, the polynucleotides and/or amplification product (e.g., amplicon) can be anchored to a polymer matrix. For example, the polymer matrix can be a hydrogel. In some embodiments, one or more of the oligonucleotides probe(s) can be modified to contain functional groups (e.g., photoreactive nucleotides) that can be used as an anchoring site to attach the polynucleotide probes and/or amplification product to a polymer matrix. In some embodiments, the nucleic acid concatemer can further comprise one or more amine-modified nucleotides. Exemplary polymer matrix that can be employed in accordance with the provided embodiments comprise those described in, for example, US 2016/0024555, US 2018/0251833, US 2016/0024555, US 2018/0251833 and US 2017/0219465, which are herein incorporated by reference in their entireties. In some examples, the scaffold can comprise oligonucleotides, polymers or chemical groups, to provide a matrix and/or support structures.

In some preferred embodiments, the nucleic acid concatemer is an amplification product, such as a rolling circle amplification (RCA) product. RCA may comprise contacting the biological sample with one or more probes to produce an RCA product (RCP).

In some embodiments, the methods disclosed herein involve the use of one or more probes or probe sets that directly or indirectly hybridize to a target nucleic acid, such as a nucleic acid analyte. In some aspects, the biological sample is contacted with a barcoded probe or barcoded probe set. In some embodiments, the barcode probe or barcoded probe set comprises one or more barcode sequences. Therefore, in some embodiments, the nucleic acid concatemer comprises one or more barcode sequences. The barcode sequence may be specific to a target nucleic acid (e.g., an analyte) in the biological sample, thereby serving to identify the target nucleic acid during downstream analyses.

In some embodiments, the one or more probes or probe sets comprise a circular probe, or a circularizable probe or probe set. In some embodiments, the circular probe or the circularizable probe or probe set is or comprises an oligonucleotide. A circularizable probe or probe set may be ligated to form a circularized probe. In some embodiments, the circular probe or the circularizable probe or probe set comprises a barcode sequence. In some embodiments, the barcode sequence of the circular probe or the circularizable probe or probe set is incorporated into the nucleic acid concatemer during amplification (e.g., RCA).

In some aspects, provided herein are methods for generating a nucleic acid concatemer, wherein the nucleic acid concatemer comprises multiple copies of an oligonucleotide probe recognition site. In some embodiments, the nucleic acid concatemer is contacted with a plurality of oligonucleotide probes that hybridize to the oligonucleotide probe recognition site. In some embodiments, the circular probe, or the circularizable probe or probe set that is incorporated into the nucleic acid concatemer during amplification comprises the oligonucleotide probe recognition site. In some embodiments, the amplification of the circular probe, or the circularizable probe or probe set results in a nucleic acid concatemer comprising multiple copies of the oligonucleotide probe recognition site. In some embodiments, the nucleic acid concatmer comprises at least two copies of the oligonucleotide probe recognition site. In some embodiments, the nucleic acid concatemer comprises about 10, 100, 1,000, or 10,000 copies of the oligonucleotide probe recognition site. In some embodiments, the nucleic acid concatemer comprises between about 10 and about 100, between about 100 and about 1,000, between about 1,000 and about 5,000, between about 5,000 and about 10,000, or more than 10,000 copies of a unit sequence. In some embodiments, the oligonucleotide probe recognition site comprises between about 10 and about 40 nucleotides in length. In some embodiments, the oligonucleotide probe recognition site comprises about 10, 11, 12, 13, 14, 15, 16, 17, 18, 19, 20, 25, 30, 35, or 40 nucleotides in length.

In some embodiments, the oligonucleotide probe recognition site comprises a first recognition sequence and a second recognition sequence. In some embodiments, the first recognition sequence and the second recognition sequence are identical (e.g., comprise the same sequence of nucleic acids and are the same length). In some embodiments, the first recognition sequence and the second recognition sequence are identical in length, but not in nucleic acid sequence. In some embodiments, the first recognition sequence and the second recognition sequence are different in length and in nucleic acid sequence. In some embodiments, the first and/or second recognition sequences are between about 5 and about 20 nucleotides in length, such as any of about 5, 10, 15, or 20 nucleotides in length. In some embodiments, the first recognition sequence and the second recognition sequences do not overlap.

In some embodiments, a probe disclosed herein is amplified through rolling circle amplification (RCA). In some embodiments, the method comprises using a circular or circularizable construct hybridized to the nucleic acid of interest to generate a circular nucleic acid. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In some embodiments, the circular nucleic acid is a construct formed using ligation. In some embodiments, the circular construct is formed using template primer extension followed by ligation. In some embodiments, the circular construct is formed by providing an insert between ends to be ligated. In some embodiments, the circular construct is formed using a combination of any of the foregoing. In some embodiments, the ligation is a DNA-DNA templated ligation. In some embodiments, the ligation is an RNA-RNA templated ligation. Exemplary RNA-templated ligation probes and methods are described in US 2020/0224244 which is incorporated herein by reference in its entirety. In some embodiments, the ligation is a RNA-DNA templated ligation. In some embodiments, a splint is provided as a template for ligation.

In some embodiments, a circularizable probe or probe set disclosed herein (e.g., a padlock probe) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693- 24700 and in US 2020/0224244, the entire contents of each of which are incorporated herein by reference, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single- stranded region, e.g. as described in Lyamichev et al. 1999. PNAS 96, 6143-6148, the entire contents of which are incorporated herein by reference, for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

Following formation of the circular nucleic acid, in some instances, an amplification primer is added. In other instances, the amplification primer is added with the primary and/or secondary probes. In some instances, the amplification primer may also be complementary to the target nucleic acid and the circularizable probe. In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a padlock probe) the circularized probe is rolling-circle amplified to generate a RCA product (e.g., amplicon) containing multiple copies of the circular.

Suitable examples of DNA polymerases that can be used include, but are not limited to: E.coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, rolling circle amplification products are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

Following amplification, the sequence of the amplicon (e.g., RCA product) or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, a sequence of the RCA product is detected using, e.g., the secondary and higher order probes and detection oligonucleotides described herein.

In some embodiments, a circularizable probe or probe set (e.g., a padlock probe) contains one or more barcodes. In some embodiments, the barcodes are bound by detectable probes, which may but do not need to be detectably labeled. In some embodiments, one or more decoding schemes are used to decode the signals, such as fluorescence, for sequence determination. Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); US 10,457,980 B2; US 2016/0369329 A1; WO 2018/026873 A1; US 2020/0080139;and US 2017/0220733 A1, all of which are incorporated by reference in their entirety. In some embodiments, these assays enable signal amplification, combinatorial decoding, and error correction schemes at the same time.

### IV. Oligonucleotide Probe Design, Hybridization, and Ligation

The present disclosure provides methods for stabilizing and/or compacting a nucleic acid concatemer for in situ analyses. A biological sample comprising a nucleic acid concatemer may be contacted with one or more oligonucleotide probes. Each oligonucleotide probe comprises a hybridizing region capable of hybridizing to an oligonucleotide probe recognition site or portion thereof present on the nucleic acid concatemer, and an overhang that does not hybridize to the nucleic acid concatemer (e.g., an oligonucleotide probe recognition site or portion thereof). The overhangs of the one or more oligonucleotide probes hybridized to different copies of the oligonucleotide probe recognition site of the nucleic acid concatemer may be connected (e.g., ligated). The connection of the overhangs of the one or more oligonucleotide probes may involve the use of a splint, wherein the ends of the overhangs (e.g., splint recognition regions) are hybridized to a splint that does not hybridize to the nucleic acid concatemer. The splint may help mediate the connection of the overhangs of the one or more oligonucleotide probes. The ends (e.g., the 3′ and/or 5′ ends) of the overhangs of the oligonucleotide probes may comprise click functional groups, which may be ligated during the connecting step. The described methods are useful for the stabilization and/or compaction of nucleic acid concatemers in a biological sample, which provides advantages for downstream in situ analyses (e.g., target sequence detection).

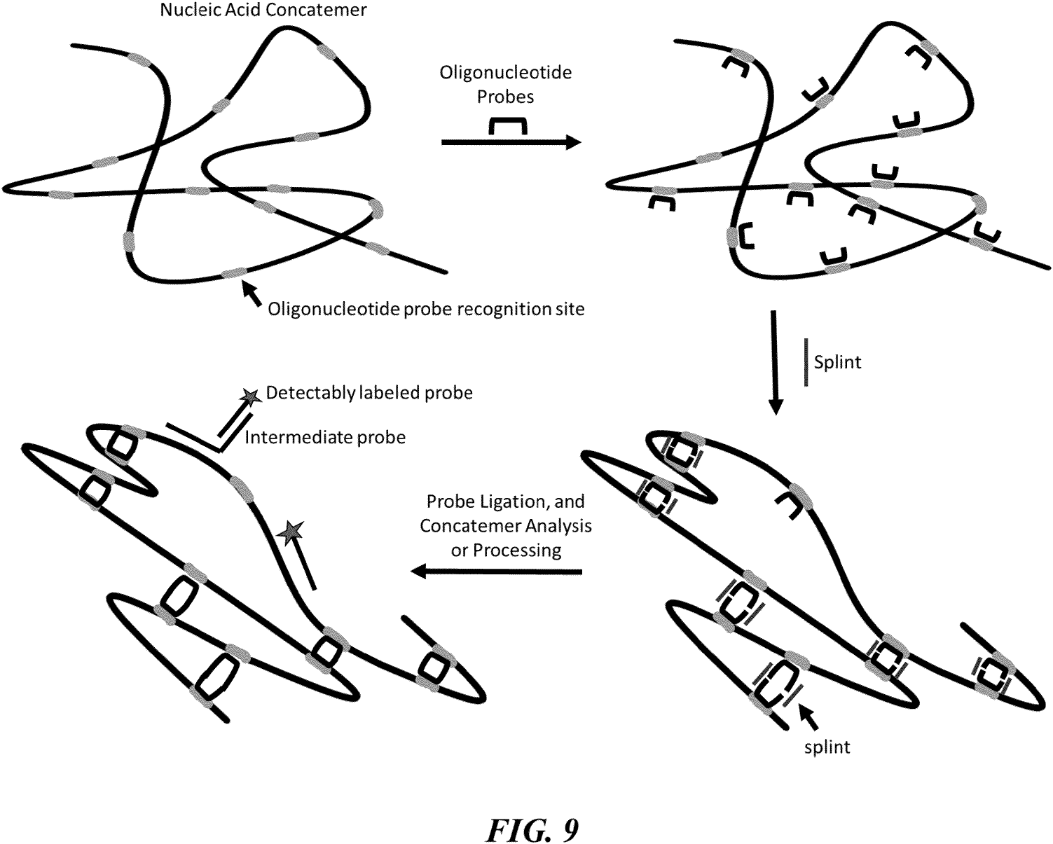

In some embodiments, the present disclosure provides methods and compositions for stabilization and/or compaction of nucleic acid concatemers (see FIG. 9 for an exemplary workflow). In some embodiments, the composition and methods disclosed herein allow for stabilization of nucleic acid concatemers comprising multiple copies of an oligonucleotide probe recognition site. In some aspects, provided herein are methods and compositions for stabilizing nucleic acid concatemers using one or more oligonucleotide probes, wherein each oligonucleotide probe comprises at least one hybridizing region complementary to the oligonucleotide probe recognition site, or a portion thereof. In some embodiments, the oligonucleotide probe further comprises at least one overhang (e.g., splint recognition region). In some embodiments, the oligonucleotide probe comprises an optional splint recognition region, and a terminal functional group (e.g., click functional group) to be used to ligate the probe to itself, to one or more additional oligonucleotide probes, or to the hydrogel matrix. In some aspects, the overhang regions are not complementary to the oligonucleotide probe recognition site or a portion thereof. In some embodiments, the oligonucleotide probe hybridizes to the oligonucleotide probe recognition site on nucleic acid concatemer at the hybridizing region. In some embodiments, splints are introduced to the nucleic acid concatemer. In some embodiments, the splints are introduced following hybridization of the oligonucleotide probes to the nucleic acid concatemer. In some aspects, the splints hybridize to the splint recognition regions of the oligonucleotide probe overhang regions, bringing the functional groups at the termini of the oligonucleotide probes into close proximity. In some embodiments, the splints are used to mediate ligation of the functional groups at the termini of the oligonucleotide probes via splint-mediated ligation. In some embodiments, the hybridization of the oligonucleotide probes to the nucleic acid concatemer followed by the subsequent ligation of the ends of the individual probes results in stabilization of the nucleic acid concatemer. In some embodiments, stabilization of the nucleic acid concatemer allows the concatemer to remain in the same configuration following rounds of in situ analysis and processing, such as in situ hybridization or sequencing. In some embodiments, stabilization of the nucleic acid concatemer allows the concatemer to remain detectable following rounds of in situ analysis and processing. In some embodiments, the oligonucleotide probes also facilitate compaction of the nucleic acid concatemer in addition to stabilization.

In some aspects, provided herein is a method for analyzing a biological sample, comprising generating a nucleic acid concatemer (e.g., a rolling circle product (RCP) generated from rolling circle amplification (RCA)) in the biological sample. In some embodiments, the nucleic acid concatemer comprises multiple copies of an oligonucleotide probe recognition site. In some embodiments, the biological sample comprising the nucleic acid concatemer is contacted with one or more oligonucleotide probes. In some embodiments, the one or more oligonucleotide probes comprise a hybridizing region capable of hybridizing to an oligonucleotide probe recognition site or portion thereof present on the nucleic acid concatemer, and an overhang that does not hybridize to the nucleic acid concatemer. In some embodiments, the overhangs of the one or more oligonucleotide probes are connected. In some embodiments, the overhangs of the one or more oligonucleotide probes hybridized to different copies of the oligonucleotide probe recognition site of the nucleic acid concatemer are connected. In some embodiments, the ends of the overhangs of the oligonucleotide probes (e.g., splint recognition regions) are hybridized to a splint during the connecting step. In some embodiments, the ends (e.g., the 5′ and/or 3′ ends) of the one or more oligonucleotide probes comprise click functional groups. In some embodiments, the connecting step comprises ligating the click functional groups of the one or more oligonucleotide probes hybridizes to different copies of the oligonucleotide probe recognition site. In some embodiments, the one or more oligonucleotide probes comprise one or more functional groups for attachment to the matrix. In some embodiments, the one or more oligonucleotide probes comprise a 5′ azide and the matrix comprises a 3′ alkyne.

In some embodiments, the first oligonucleotide probe comprises (i) a 5′ azide and a 5′ sequence that hybridizes to a first splint, and (ii) a 3′ alkyne and a 3′ sequence that hybridizes to a second splint, the second oligonucleotide probe comprises (i) a 3′ alkyne and a 3′ sequence that hybridizes to the first splint, and (ii) a 5′ azide and a 5′ sequence that hybridizes to the second splint, and upon hybridization of the first and second oligonucleotide probes to the first and second splints, the 5′ azide of the first oligonucleotide and the 3′ alkyne of the second oligonucleotide are in proximity for click reaction, and the 3′ alkyne of the first oligonucleotide and the 5′ azide of the second oligonucleotide are in proximity for click reaction.

In some embodiments, disclosed herein is a splint comprising two hybridizing regions complementary to the splint recognition regions on the oligonucleotide probes or a portion thereof. In some embodiments, the splint may be between about 5 and about 50 nucleotides in length. In some embodiments, disclosed herein is an oligonucleotide probe set comprising a first oligonucleotide probe and a second oligonucleotide probe. Each oligonucleotide probe comprises one hybridizing region complementary to the oligonucleotide probe recognition site or a portion thereof on a nucleic acid concatemer, and an overhang that is not complementary to the nucleic acid concatemer. Optionally, the overhang comprises a splint recognition region complementary to the splint or a portion thereof. In some embodiments, the first oligonucleotide probe comprises a splint recognition region on the 3′ end of the probe and the second oligonucleotide probe comprises a splint recognition region on the 5′ end of the probe. The 3′ end of the first splint recognition region on the first oligonucleotide probe comprises a functional group (e.g., a click functional group) that may be ligated to a functional group on the 5′ end of the second splint recognition region on the second oligonucleotide probe. In some embodiments, the first oligonucleotide probe comprises a hybridizing region adjacent to an overhang (e.g., an optional first splint recognition region). In some embodiments, the second oligonucleotide probe comprises a hybridizing region adjacent to an overhang (e.g., an optional second splint recognition region). The hybridizing regions can be identical or different. In some embodiments, the hybridizing regions are complementary to the oligonucleotide probe recognition site or a portion thereof on the nucleic acid concatemer. In some embodiments, the overhangs do not hybridize with the nucleic acid concatemer, and are optionally complementary to a splint.

In some embodiments, disclosed herein is an oligonucleotide probe in linear form. In some embodiments, the oligonucleotide probe comprises a first overhang (e.g., a first splint recognition region) on the 5′ end of the probe flanked by a hybridizing region, and a second overhang (e.g., a second splint recognition region) on the 3′ end of the probe. In some embodiments, the hybridizing regions are complementary to the oligonucleotide probe recognition site or a portion thereof on the nucleic acid concatemer. In some embodiments, the hybridizing region comprises between about five and thirty nucleotides in length. In some embodiments, the overhangs (e.g., splint recognition regions) comprise between about three and twenty-five nucleotides in length. The set of probes may comprise probes that have the same recognition region or probes with different recognition regions.

In some embodiments, disclosed herein are oligonucleotide probes hybridized to a nucleic acid concatemer and connected to a hydrogel matrix. In some embodiments, a first oligonucleotide probe is hybridized to a first copy of the oligonucleotide probe recognition site on the concatemer. In some embodiments, the first oligonucleotide probe is ligated to a second oligonucleotide probe via functional groups present at one end of the first oligonucleotide probe and the first end of the second oligonucleotide probe. Optionally, a first splint is hybridized to overhangs via the splint recognition regions of the first and second oligonucleotide probes to mediate the ligation of the probes. In some embodiments, the second oligonucleotide probe does not hybridize to the nucleic acid concatemer via its hybridizing region and is not in direct contact with the nucleic acid concatemer. In some embodiments, the second oligonucleotide probe is ligated to a third oligonucleotide probe via functional groups at the second end of the second oligonucleotide probe not ligated to the first oligonucleotide probe and the first end of the third oligonucleotide probe. Optionally, a second splint is hybridized to overhangs via the splint recognition regions of the first and second oligonucleotide probes to mediate the ligation of the probes. In some embodiments, the hybridizing region of the third oligonucleotide probe is hybridized to a second copy of the oligonucleotide probe recognition site on the concatemer. In some embodiments, the third oligonucleotide probe is additional ligated to the hydrogel matrix in which the concatemer is embedded at the end of the overhang that is not ligated to the second oligonucleotide probe.

In some embodiments, in the connecting step, the ends of an oligonucleotide probe are connected, wherein the oligonucleotide probe comprises (i) hybridizing regions that hybridize to different copies of the oligonucleotide probe recognition site and (ii) a linker (e.g., a flexible spacer) between the hybridizing regions that does not hybridize to the nucleic acid concatemer. In some embodiments, the linker is a nucleic acid linker. In some embodiments, the linker comprises a non-nucleic acid component. In some embodiments, the linker can be a chemical linker, such as a carbon spacer group of different lengths, e.g., C3-18 spacers (e.g., structures with 3-18 consecutive carbon atoms, such as C3 spacer) and ethylene glycol multimer spacers (e.g., spacer 18 (hexa-ethyleneglycol spacer). In some embodiments, the linker is a tetra-ethylene glycol (TEG) linker. Exemplary linkers include, but are not limited to, Amino Linker C6, Amino Linker C12, Spacer C3, Spacer C6, Spacer C12, Spacer 9, and Spacer 18. In some embodiments, the linker is a nucleic acid comprising a sequence that can be recognized by a detectably labeled probe, such as a fluorescently labeled oligonucleotide. In some instances, the nucleic acid linker sequence is selected such that it is not substantially complementary to a sequence in a nucleic acid concatemer or a hybridizing region of a stabilization/compaction probe disclosed herein. In some embodiments, the linker is between about 0 and about 200 nucleotides in length, such as 1, 2, 3, 4, 5, 6, 7, 8, 9, 10, 11, 12, 13, 14, 15, 16, 17, 18, 19, 20 or more nucleotides. In some embodiments, the first oligonucleotide probe comprises a 5′ azide and a 5′ sequence that hybridizes to a splint, the second oligonucleotide probe comprises a 3′ alkyne and a 3′ sequence that hybridizes to the splint, and upon hybridization of the first and second oligonucleotide probes to the splint, the 5′ azide and 3′ alkyne are in proximity for click reaction.

In some embodiments, disclosed herein is an oligonucleotide probe in linear form. In some embodiments, the oligonucleotide probe comprises a first overhang (e.g., a first splint recognition region) at its 5′ end with a first hybridizing region, and a second overhang (e.g., a second splint recognition region) at its 3′ end flanked by a second hybridizing region. In some embodiments, the hybridizing regions are complementary to the oligonucleotide probe recognition site or a portion thereof on the nucleic acid concatemer. In some embodiments, the first and second hybridizing regions are separated by a linker region that does not hybridize to either the nucleic acid concatemer or the splint probe.

In some embodiments, disclosed herein is an oligonucleotide probe hybridized to a nucleic acid concatemer. In some embodiments, the first hybridizing region of the oligonucleotide probe is hybridized to a first copy of the oligonucleotide probe recognition site on the concatemer, and the second hybridizing region of the same oligonucleotide probe is hybridized to a second copy of the oligonucleotide probe recognition site on the concatemer. In some embodiments, the functional group at the 5′ end of the oligonucleotide probe is ligated to the functional group at the 3′ end of the same oligonucleotide probe in a connecting step. Optionally, the ligation is mediated by a splint hybridized to the overhangs of the oligonucleotide probe.

In some embodiments, disclosed herein is an oligonucleotide probe comprising a first hybridizing region and a second hybridizing region separated by a linker region. The sequence of the hybridizing regions may be identical or may be different. In some embodiments, disclosed herein is an oligonucleotide probe in linear form, comprising a first hybridizing region and a second hybridizing region separated by a linker region. In some embodiments, the hybridizing regions are complementary to the oligonucleotide probe recognition site, or a portion thereof, on the nucleic acid concatemer. In some embodiments, the linker region is not complementary to the nucleic acid concatemer. In some embodiments, the individual hybridizing regions are between about five and thirty nucleotides in length. The sequence of the hybridizing regions may be identical or may be different. In some embodiments, disclosed herein is an oligonucleotide probe hybridized to a nucleic acid concatemer. In some embodiments, the first hybridizing region of the oligonucleotide probe is hybridized a first copy of the oligonucleotide probe recognition site on the concatemer, and the second hybridizing region of the same oligonucleotide probe is hybridized to a second copy of the oligonucleotide probe recognition site on the concatemer. Optionally, only one hybridizing region of the oligonucleotide probe is hybridized to the nucleic acid concatemer.

In some embodiments, disclosed herein is an oligonucleotide probe set in linear form. A first oligonucleotide probe comprises a first hybridizing region and a second hybridizing region capable of hybridizing to an oligonucleotide recognition site on a nucleic acid concatemer, separated by a linker region. A second oligonucleotide probe comprises a first hybridizing region and a second hybridizing region, capable of hybridizing to an oligonucleotide recognition site on a nucleic acid concatemer, separated by a linker region. The individual hybridizing regions are independently between five and thirty nucleotides in length. The linker regions may or may not be complementary to one another. The first hybridizing region and second hybridizing regions of the first oligonucleotide probe may be identical or may be different. The first hybridizing region and the second hybridizing region of the second oligonucleotide probe may be identical or may be different. Optionally, the linker region of the first oligonucleotide probe and the linker region of the second oligonucleotide probe may be complementary in sequence. Further optionally, the linker region of the first oligonucleotide probe and the linker region of the second oligonucleotide probe may not be complementary in sequence. In some embodiments, the first nucleotide probe comprises a first hybridizing region adjacent to a linker region, and a second hybridizing region. In some embodiments, the second nucleotide probe comprises a first hybridizing region adjacent to a linker region and a second hybridizing region. The second oligonucleotide probe is oriented so the termini are adjacent to the junctions of the linker region and hybridizing regions of the first oligonucleotide probe. In some embodiments, the first nucleotide probe comprises a first hybridizing region adjacent to a linker region and a second hybridizing region. In some embodiments, the second nucleotide probe comprises a first hybridizing region adjacent to a linker region and a second hybridizing region. The second oligonucleotide probe is oriented so that its linker region is aligned with the linker region of the first oligonucleotide probe. The linker regions may hybridize to one another.

In some aspects, provided herein is a method for analyzing a biological sample, comprising (a) contacting the biological sample with one or more oligonucleotide probes, wherein: a nucleic acid concatemer comprising multiple copies of an oligonucleotide probe recognition site is generated in the biological sample, and each oligonucleotide probe comprises (i) a hybridizing region that hybridizes to the oligonucleotide probe recognition site or a portion thereof, and (ii) an overhang that does not hybridize to the nucleic acid concatemer; and (b) connecting the overhangs of the one or more oligonucleotide probes hybridized to different copies of the oligonucleotide probe recognition site.

In some aspects, provided herein is a method for analyzing a biological sample, comprising contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; contacting the biological sample with a first oligonucleotide probe and a second oligonucleotide probe each comprising (i) a hybridizing region complementary to the oligonucleotide probe recognition site or a portion thereof, and (ii) an overhang that does not hybridize to the RCP, wherein the hybridizing regions of the first and second oligonucleotide probes are hybridized to different copies of the oligonucleotide probe recognition site; contacting the biological sample with a splint that hybridizes to the overhangs of the first and second oligonucleotide probes; and ligating the overhangs hybridized to the splint, thereby forming a ligated product of the first and second oligonucleotide probes hybridized to different copies of the oligonucleotide probe recognition site in the RCP. In some embodiments, the ligating step comprises click ligation. In some embodiments, the first and second oligonucleotide probes comprise a 5′ azide and a 3′ alkyne, respectively. In some embodiments, the biological sample is embedded in a matrix comprising a 3′ alkyne.

In some aspects, provided herein is a method for analyzing a biological sample, comprising contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; contacting the biological sample with a first oligonucleotide probe and a second oligonucleotide probe each comprising (i) a hybridizing region complementary to the oligonucleotide probe recognition site or a portion thereof, and (ii) a 5′ overhang and a 3′ overhang that do not hybridize to the RCP, wherein the hybridizing regions of the first and second oligonucleotide probes are hybridized to different copies of the oligonucleotide probe recognition site; contacting the biological sample with a first splint that hybridizes to the 5′ overhang of the first oligonucleotide probe and the 3′ overhang of the second oligonucleotide probe, and with a second splint that hybridizes to the 3′ overhang of the first oligonucleotide probe and the 5′ overhang of the second oligonucleotide probe; and ligating the overhangs hybridized to the first splint and the overhangs hybridized to the second splint, thereby forming a ligated product of the first and second oligonucleotide probes hybridized to different copies of the oligonucleotide probe recognition site in the RCP. In some embodiments, the ligating step comprises click ligation. In some embodiments, the first and second oligonucleotide probes comprise a 5′ azide and a 3′ alkyne, respectively. In some embodiments, the biological sample is embedded in a matrix comprising a 3′ alkyne.

In some aspects, provided herein is a method for analyzing a biological sample comprising contacting the biological sample with a circular probe or circularizable probe or probe set comprising a sequence that hybridizes to a target nucleic acid in the biological sample and a sequence complementary to an oligonucleotide probe recognition site, wherein a rolling circle amplification product (RCP) is generated in the biological sample using the circular probe or a circularized probe generated from the circularizable probe or probe set as a template, wherein the RCP comprises multiple copies of the oligonucleotide probe recognition site; contacting the biological sample with an oligonucleotide probe comprising (i) a first hybridizing region and a second hybridizing region, each of which is complementary to the oligonucleotide probe recognition site or a portion thereof, (ii) a linker between the first and second hybridizing regions, and (iii) a 5′ overhang and a 3′ overhang, wherein the linker and the overhangs do not hybridize to the RCP, and wherein the first and second hybridizing regions are hybridized to different copies of the oligonucleotide probe recognition site; contacting the biological sample with a splint that hybridizes to the overhangs of the oligonucleotide probe; and ligating the overhangs hybridized to the splint, thereby forming a ligated product of the oligonucleotide probe hybridized to different copies of the oligonucleotide probe recognition site in the RCP. In some embodiments, the ligating step comprises click ligation. In some embodiments, the first and second oligonucleotide probes comprise a 5′ azide and a 3′ alkyne, respectively. In some embodiments, the biological sample is embedded in a matrix comprising a 3′ alkyne.

In other aspects, provided herein is a method for analyzing a biological sample, comprising contacting the biological sample with a first oligonucleotide probe and a second oligonucleotide probe, wherein a nucleic acid concatemer comprising multiple copies of an oligonucleotide probe recognition site is generated in the biological sample, the oligonucleotide probe recognition site comprises a first recognition sequence and a second recognition sequence, the first oligonucleotide probe comprises: (i) a 5′ hybridizing region that hybridizes to the first recognition sequence or portion thereof, (ii) a linker that does not hybridize to the nucleic acid concatemer, and (iii) a 3′ hybridizing region that hybridizes to the first recognition sequence or portion thereof, the second oligonucleotide probe comprises: (i) a 5′ hybridizing region that hybridizes to the second recognition sequence or portion thereof, (ii) a linker that does not hybridize to the nucleic acid concatemer, and (iii) a 3′ hybridizing region that hybridizes to the second recognition sequence or portion thereof, and the first and second oligonucleotide probes are each hybridized to different copies of the oligonucleotide probe recognition site.

Certain features and steps of the methods disclosed herein are described with additional detail in the sections below. The modular discussion of certain features and steps is not intended to limit the scope of the methods described herein, and using the teachings provided herein one can readily combine various modularly described features and steps to arrive at the full scope of the methods provided herein.

### A. Oligonucleotide Probes

### (i) Probe Sequences

In some aspects, the methods described herein comprise contacting a biological sample with one or more oligonucleotide probes. The oligonucleotide probes provided herein are capable of hybridizing to specific sites on a nucleic acid concatemer (e.g., oligonucleotide probe recognition sites) in the biological sample.

In some embodiments, the biological sample is in contact with the one or more oligonucleotide probes prior to or at the initiation of the generation of the nucleic acid concatemer (e.g., a rolling circle product (RCP)). In some embodiments, the biological sample is in contact with the one or more oligonucleotide probes during the generation of the nucleic acid concatemer. In some embodiments, the biological sample is in contact with the one or more oligonucleotide probes after the generation of the nucleic acid concatemer. For example, the nucleic acid concatemer may have about ten copies of an oligonucleotide recognition site at the time one or more oligonucleotide probes are added to the biological sample. In some embodiments, the nucleic acid can be continued to be generated in the biological sample (e.g., by rolling circle amplification (RCA)) to extend the nucleic acid concatemer (e.g., RCP) to 100, 1000, or more copies of the oligonucleotide recognition site after contacting the biological sample with one or more oligonucleotide probes.

In some embodiments, the oligonucleotide probe comprises a hybridizing region capable of hybridizing with a nucleic acid concatemer. In some embodiments, the hybridizing region of the oligonucleotide probe hybridizes with an oligonucleotide probe recognition site on a nucleic acid concatemer, or a portion thereof. In some embodiments, the hybridizing region comprises a nucleic acid sequence of between about 5 and about 15 base pairs in length. In some embodiments, the hybridizing region comprises a nucleic acid sequence of about 10 base pairs in length.

In some embodiments, the oligonucleotide probe comprises at least one hybridizing region. In some embodiments, the oligonucleotide probe comprises two hybridizing regions. In some embodiments, the hybridizing regions are identical in sequence. In some embodiments, the hybridizing regions comprise different sequences. In some embodiments, the hybridizing regions are identical in length. In some embodiments, the hybridizing regions are of different length. In some embodiments, the hybridizing regions are identical in length but comprise different sequences. In some embodiments, one end of the oligonucleotide probe is in the hybridizing region and the other end is not. In some embodiments, the oligonucleotide probe comprises two hybridizing regions. In some embodiments, the ends of the oligonucleotide probe are in one of the two hybridizing regions. In some embodiments, the hybridizing regions do not overlap. In some embodiments, the at least two hybridizing regions are positioned directly adjacent to one another on the oligonucleotide probe. In some embodiments, the hybridizing regions do not hybridize to each other.

In some embodiments, the oligonucleotide probe further comprises an overhang. In some embodiments, the overhang is not complementary (e.g., does not hybridize) to an oligonucleotide probe recognition site, or a portion thereof, on a nucleic acid concatemer. In some embodiments, the oligonucleotide probe comprises at least one overhang. In some embodiments, the oligonucleotide probe comprises one or more overhangs, such as any of two overhangs, three overhangs, or four overhangs. In some embodiments, the overhangs of the oligonucleotide probes are independently between about 3 and about 25 nucleotides in length, such as between any of about 3 and about 10 nucleotides in length, about 8 and about 15 nucleotides in length, about 12 and 20 nucleotides in length, and 18 and about 25 nucleotides in length. In some embodiments, the overhangs independently comprise less than about 25 nucleotides in length, such as less than about 24, 23, 22, 21, 20, 16, 15, 14, 13, 12, 11, 10, 9, 8, 7, 6, 5, 4, 3, or 2 nucleotides in length. In some embodiments, the overhangs are independently about 6, about 8, about 10, about 12, about 14, or about 16 nucleotides in length. In some embodiments, the end (e.g., the 3′ end or the 5′ end) of the oligonucleotide probe comprises the overhang. In some embodiments, both ends of the oligonucleotide probe comprise the overhangs. In some embodiments, the oligonucleotides probe comprises two or more overhangs. In some embodiments, the overhangs of the same oligonucleotide probe do not hybridize to each other.

In some embodiments, the overhangs comprise a splint recognition region. In some embodiments, the splint recognition region is not complementary (e.g., does not hybridize) to an oligonucleotide probe recognition site, or a portion thereof, on a nucleic acid concatemer. In some embodiments, the splint recognition region is capable of hybridizing with a splint (e.g., any of the splints described herein). In some embodiments, the splint is in contact with the splint recognition region of the oligonucleotide probes following hybridization of the oligonucleotide probes to the oligonucleotide probe recognition site on the nucleic acid concatemer. In some embodiments, the splint recognition region comprises the entirely of the overhang. In some embodiments, the splint recognition region is a portion of the overhang. In some embodiments, the overhang comprises one or more splint recognition regions. In some embodiments, the splint recognition region comprises between about 3 and about 25 nucleotides in length, such as between any of about 3 and about 10 nucleotides in length, about 8 and about 15 nucleotides in length, about 12 and 20 nucleotides in length, and 18 and about 25 nucleotides in length. In some embodiments, the splint recognition region comprises less than about 25 nucleotides in length, such as less than about 24, 23, 22, 21, 20, 16, 15, 14, 13, 12, 11, 10, 9, 8, 7, 6, 5, 4, 3, or 2 nucleotides in length. In some embodiments, the splint recognition region comprises about 6, about 8, about 10, about 12, about 14, or about 16 nucleotides in length.

In some embodiments, the oligonucleotide probe further comprises a linker. In some embodiments, the linker is not complementary (e.g., does not hybridize) to an oligonucleotide probe recognition site, or a portion thereof, on a nucleic acid concatemer. In some embodiments, the linker comprises a nucleic acid sequence that is any of about 19, 18, 17, 16, 15, 14, 13, 12, 11, 10, 9, 8, 7, 6, 5, 4, 3, 2, or 1 base pairs in length. In some embodiments, the linker comprises about 1, 2, 3, 4, 5, 6, 7, 8, 9, 10, 11, 12, 13, 14, 15, 16, 17, 18, 19, or 20 or more base pairs in length. In some embodiments, the oligonucleotide probe comprises one, two, three, or four linkers. In some embodiments, the linkers regions are identical in sequence. In some embodiments, the linkers comprise different sequences. In some embodiments, the linkers are identical in length. In some embodiments, the linkers are of different length. In some embodiments, the linkers are identical in length but comprises different sequences. In some embodiments, the linkers do not overlap.

In some embodiments, the linker position on the oligonucleotide probe is relative to that of the two or more hybridizing regions of the oligonucleotide probe. In some embodiments, the linker is positioned between two hybridizing regions of the oligonucleotide probe. In some embodiments, the ends of the oligonucleotide probe are in the linker. In some embodiments, the linker comprises a stem-loop structure, and the ends of the oligonucleotide probe are in the stem of the stem-loop structure. For example, the oligonucleotide probe may comprise two hybridizing regions positioned adjacent to one another, with a linker flanking the hybridizing regions on one or bother ends of the oligonucleotide probe. In some embodiments, the linker is flanked on its 5′ end, 3′ end, or on both its 5′ end and 3′ end, by a hybridizing region.

In some embodiments, the one or more oligonucleotide probes may further comprise a detectable label. In some embodiments, the one or more oligonucleotide probes are covalently labeled with a detectable label. In some embodiments, the one or more oligonucleotide probes are covalently labeled with a detectable label via an optional linker (e.g., any suitable linker). In some embodiments, the detectable label is a fluorophore. In some embodiments, the one or more oligonucleotide probes are not covalently labeled with a detectable label.

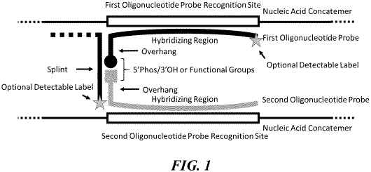

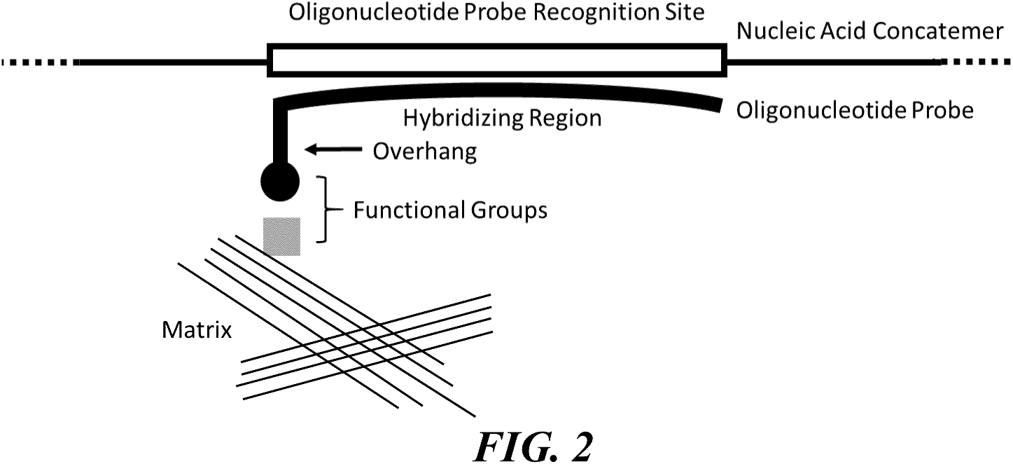

FIG. 1 depicts a first exemplary design of an oligonucleotide probe provided herein. In some embodiments, stabilization of a nucleic acid concatemer relies upon one or more unique oligonucleotide probes. In some embodiments, a first oligonucleotide probe comprises a hybridizing region complementary to the oligonucleotide probe recognition site on the nucleic acid concatemer adjacent to an overhang region on either the 5′ or 3′ end of the probe. In some embodiments, the overhang region comprises an optional first splint recognition region and a functional group (e.g., a click functional group) at the terminal end of the oligonucleotide probe. In some embodiments, a second oligonucleotide probe comprises a hybridizing region complementary to the oligonucleotide probe recognition site on the nucleic acid concatemer adjacent to an overhang on the 3′ or 5′ end of the probe. In some embodiments, the overhang region comprises an optional second splint recognition region and a functional group on the terminal end. In some embodiments, the hybridizing regions on the first and second oligonucleotide probes have the same sequence. In some embodiments, the hybridizing regions on the first and second oligonucleotide probes have different sequences. In some embodiments, the hybridizing regions of one or more oligonucleotide probes do not hybridize to each other. In some embodiments, the overhang regions on the first and second probes are on the 5′ and 3′ ends respectively, or on the 3′ and 5′ ends respectively. In some embodiments, the overhangs of one or more oligonucleotide probes are independently between about three and twenty-five nucleotides in length. In some embodiments, the functional groups at the termini of the probes at the overhang regions are click functional groups. In some embodiments, the functional groups at the termini of the probes at the overhang regions are not click functional groups. In some embodiments, one or more of the oligonucleotide probes are covalently labeled with a detectable label via an optional linker. In some embodiments, one or more of the oligonucleotide probes are not covalently labeled with a detectable label via an optional linker. FIG. 2 depicts one or more molecules of the first and/or second oligonucleotide probes can be linked to a matrix, such as a hydrogel matrix embedding the sample. The overhangs of the first and/or second oligonucleotide probes can be enzymatically and/or chemically (e.g., via click chemistry) linked to one or more functional groups or molecules in the matrix.

Exemplary first and second oligonucleotide probes can include /5AzideN/ GGCTTACGCCCGTTCCTACCGCCCTAATTAT (SEQ ID NO: 4) and CGTTCCTACCGCCCTAATTATCAAGAACGTA /350ctdU/ (SEQ ID NO: 5), in which CGTTCCTACCGCCCTAATTAT (SEQ ID NO: 1) is a hybridizing region, GGCTTACGCC (SEQ ID NO: 2) is the 5′ overhang of the first oligonucleotide probe, and CAAGAACGTA (SEQ ID NO: 3) is the 3′ overhang of the second oligonucleotide probe. A splint GGCGTAAGCCTACGTTCTTG (SEQ ID NO: 6), optionally with a delectable label, can be used to ligate the overhangs.

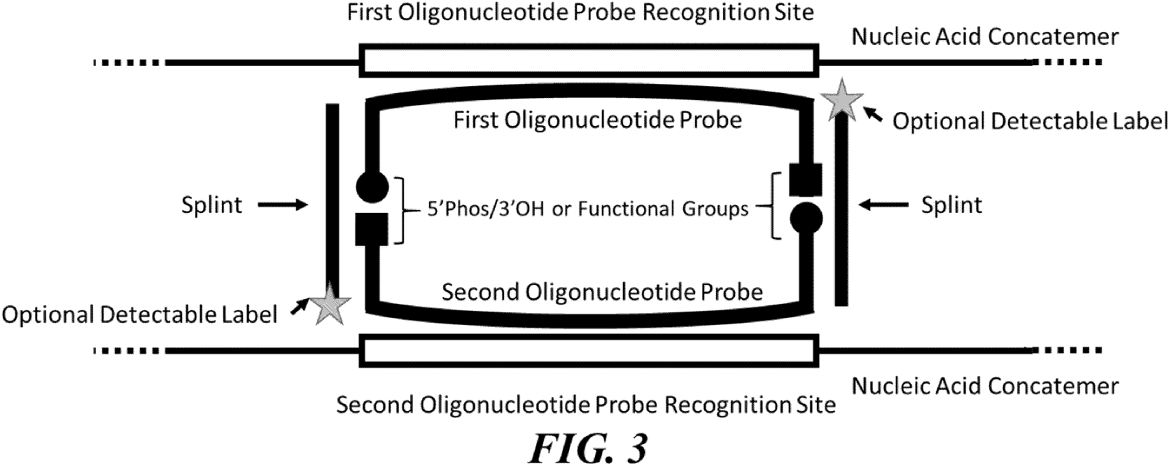

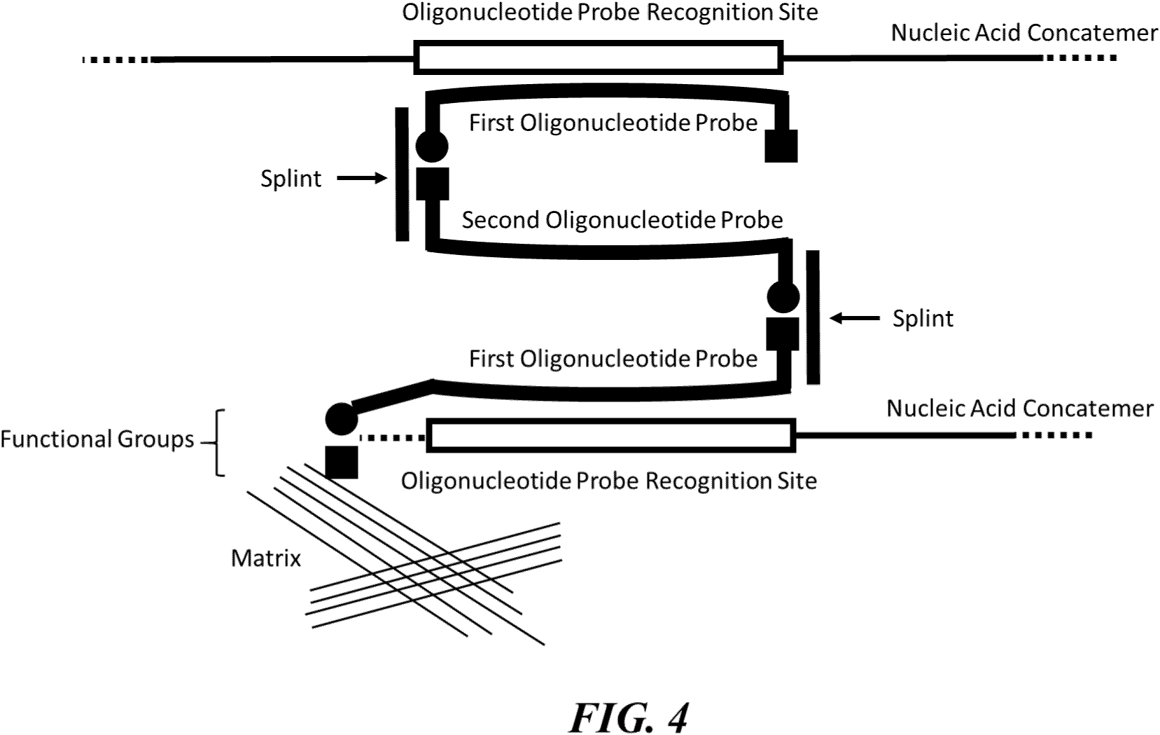

FIG. 3 depicts a second exemplary design of an oligonucleotide probe provided herein. In some embodiments, stabilization of a nucleic acid relies upon one or more oligonucleotide probes (e.g., an oligonucleotide probe set). In some embodiments, the oligonucleotide probes of an oligonucleotide probe set are identical. In some embodiments, the oligonucleotide probes of an oligonucleotide probe set are different. In some embodiments, the oligonucleotide probe comprises a hybridizing region flanked on both ends with overhang regions. In some embodiments, the hybridizing regions do not hybridize to each other. In some embodiments, the 5′ overhang region comprises an optional first splint recognition region and a functional group at the terminal end. In some embodiments, the 3′ overhang region comprises an optional second splint recognition region and a functional group at the terminal end. In some embodiments, the optional first and second splint recognition regions do not have the same sequence. In some embodiments, the functional groups present at the 5′ and 3′ ends of the oligonucleotide probe are not the same. In some embodiments, the functional groups at the ends of the oligonucleotide probe are click functional groups. In some embodiments, the click functional groups are an azide at the 5′ end and an alkyne at the 3′ end of the oligonucleotide probe. In some embodiments, the functional groups present at the 5′ and 3′ ends of the oligonucleotide probe are not click functional groups. In some embodiments, the oligonucleotide probe is covalently labeled with a detectable label via an optional linker. In some embodiments, the oligonucleotide probes is not covalently labeled with a detectable label via an optional linker. In some embodiments, the overhang regions of the oligonucleotide probe do not hybridize to each other. FIG. 4 depicts one or more molecules of the first and/or second oligonucleotide probes can be linked to a matrix, such as a hydrogel matrix embedding the sample. The overhangs of the first and/or second oligonucleotide probes can be enzymatically and/or chemically (e.g., via click chemistry) linked to one or more functional groups or molecules in the matrix.

An exemplary oligonucleotide probe can include

/5AzideN/GGCTTACGCCCGTTCCTACCGCCCTAATTATCAAGAACGTA /350ctdU/ (SEQ ID NO: 7)

, in which

GGTTCCTACCGCCCTAATTAT (SEQ ID NO: 1)

is a hybridizing region,

GGCTTACGCC (SEQ ID NO: 2)

is the 5′ overhang of the oligonucleotide probe, and

CAAGAACGTA (SEQ ID NO: 3)

is the 3′ overhang of the oligonucleotide probe. A splint

GGCGTAAGCCTACGTTCTTG (SEQ ID NO: 6)

, optionally with a delectable label, can be used to ligate the overhangs. Two molecules of this exemplary oligonucleotide probe can be used to stabilize and/or compact an RCP as shown in FIG. 3.

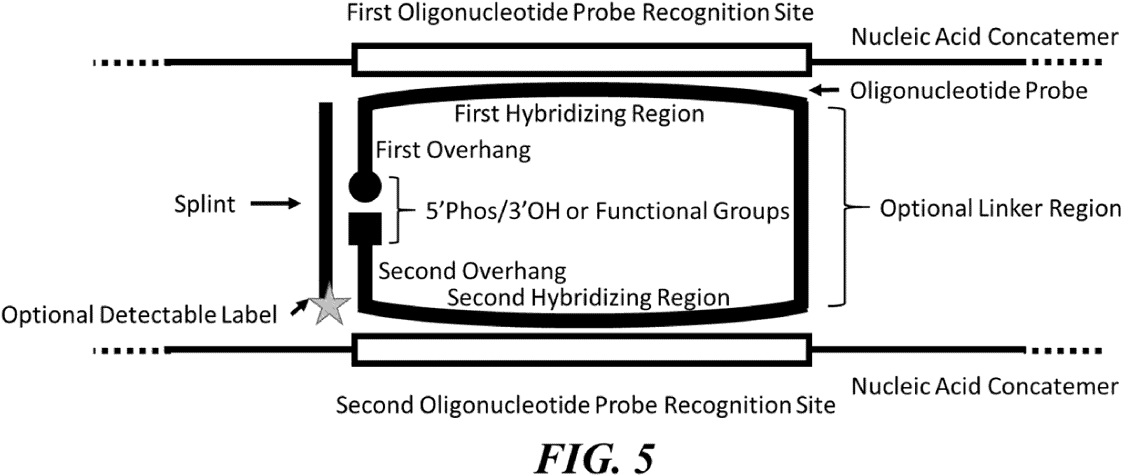

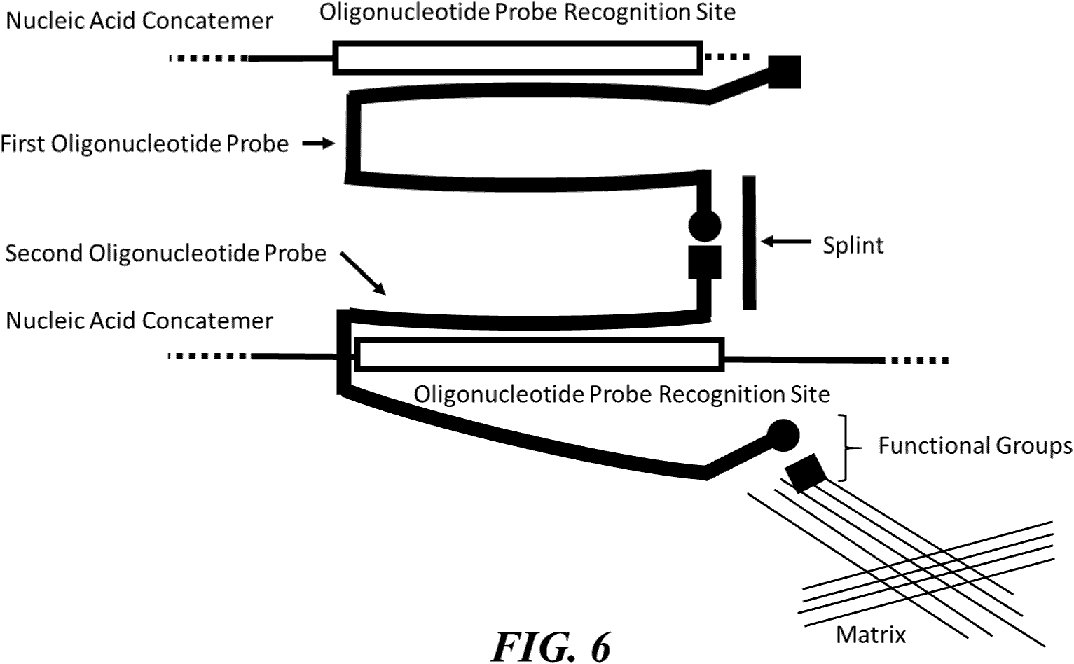

FIG. 5 depicts a third exemplary design of an oligonucleotide probe provided herein. In some embodiments, stabilization of a nucleic acid relies upon one or more oligonucleotide probes (e.g., an oligonucleotide probe set). In some embodiments, the oligonucleotide probe comprises a first overhang region on the 5′ end adjacent to a first hybridizing region, followed by a linker sequence and a second hybridizing region with a second overhang region on the 3′ end. In some embodiments, the two hybridizing regions do not hybridize to each other. In some embodiments, the linker region does not hybridize to the nucleic acid concatemer. In some embodiments, the linker region is between about 0 and about 200 nucleotides in length. In some embodiments, the 5′ overhang region comprises an optional first splint recognition region and a functional group at the terminal end. In some embodiments, the 3′ overhang region comprises an optional second splint recognition region and a functional group at the terminal end. In some embodiments, the first and second splint recognition regions do not have the same sequence. In some embodiments, the functional groups present at the 5′ and 3′ ends of the oligonucleotide probe are not the same. In some embodiments, the functional groups at the ends of the oligonucleotide probe are click functional groups. In some embodiments, the click functional groups are an azide at the 5′ end and an alkyne at the 3′ end of the oligonucleotide probe. In some embodiments, the functional groups present at the 5′ and 3′ ends of the oligonucleotide probe are not click functional groups. In some embodiments, the oligonucleotide probe is covalently labeled with a detectable label via an optional linker. In some embodiments, the oligonucleotide probes is not covalently labeled with a detectable label via an optional linker. In some embodiments, the overhang regions of the oligonucleotide probe do not hybridize to each other. FIG. 6 depicts one or more molecules of the oligonucleotide probe can be linked to a matrix, such as a hydrogel matrix embedding the sample. The overhangs of the oligonucleotide probe can be enzymatically and/or chemically (e.g., via click chemistry) linked to one or more functional groups or molecules in the matrix.

An exemplary oligonucleotide probe can include /5AzideN/ GGCTTACGCCCGTTCCTACCGCCCTAATTATTTTCGTTCCTACCGCCCTAATTATCAAGA ACGTA /350ctdU/ (SEQ ID NO: 8), in which CGTTCCTACCGCCCTAATTAT (SEQ ID NO: 1) is a hybridizing region, GGCTTACGCC (SEQ ID NO: 2) is the 5′ overhang of the oligonucleotide probe, CAAGAACGTA (SEQ ID NO: 3) is the 3′ overhang of the oligonucleotide probe, and TTT is a linker between the two copies of the hybridizing region. A splint GGCGTAAGCCTACGTTCTTG (SEQ ID NO: 6), optionally with a delectable label, can be used to ligate the overhangs. This exemplary oligonucleotide probe can be used to stabilize and/or compact an RCP as shown in FIG. 5.

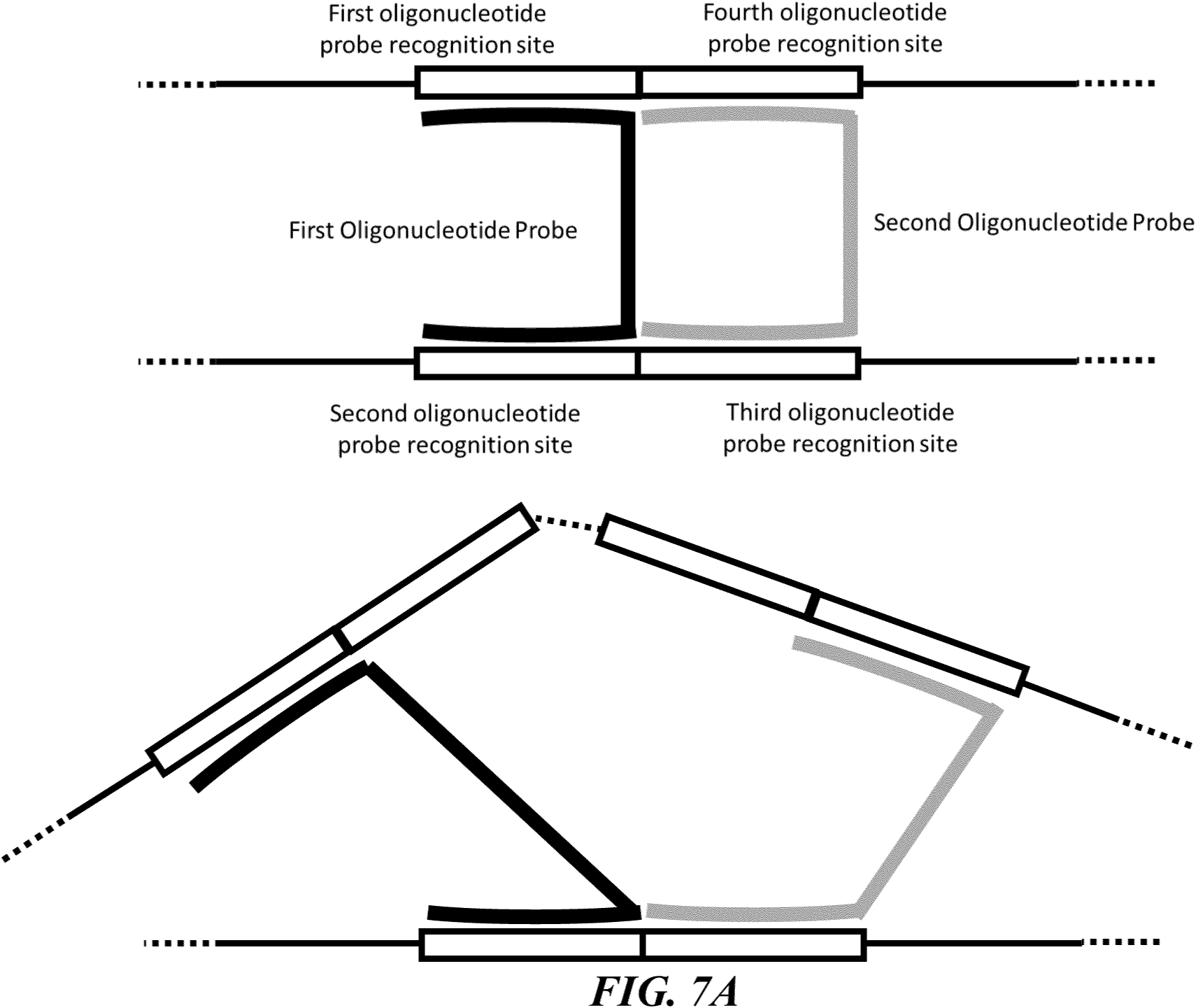

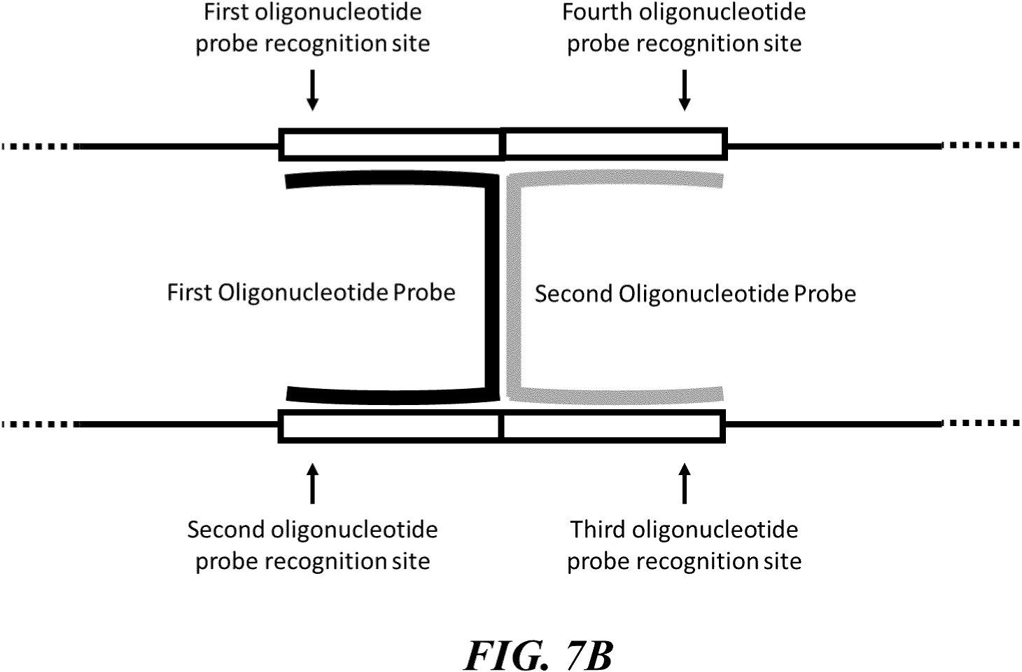

FIG. 7A depicts a fourth exemplary design of an oligonucleotide probe provided herein. In some embodiments, stabilization of a nucleic acid relies upon one or more oligonucleotide probes (e.g., an oligonucleotide probe set). In some embodiments, the oligonucleotide probes are identical. In some embodiments, the oligonucleotide probes are different. In some embodiments, the oligonucleotide probe comprises a first hybridizing region complementary to the oligonucleotide probe recognition site on the nucleic acid at the 5′ end of the probe, adjacent to a linker, and followed by a second hybridizing region complementary to the oligonucleotide probe recognition site on the nucleic acid at the 3′ end of the probe. In some embodiments, the first and second hybridizing regions have the same sequence. In some embodiments, the first and second hybridizing regions have different sequences. In some embodiments, the two hybridizing regions do not hybridize to each other. In some embodiments, the linker region does not hybridize to the nucleic acid concatemer. In some embodiments, the linker region is between about 0 and about 200 nucleotides in length. In some embodiments, the oligonucleotide probe is covalently labeled with a detectable label via an optional linker. In some embodiments, the oligonucleotide probes is not covalently labeled with a detectable label via an optional linker. In some embodiments, the overhang regions of the oligonucleotide probe do not hybridize to each other. In some embodiments, the hybridizing regions of two oligonucleotide probes hybridize to adjacent sequences in a nucleic acid concatemer (e.g., as shown in FIG. 7A, upper panel). In some embodiments, the hybridizing regions of two oligonucleotide probes hybridize to non-adjacent sequences in a nucleic acid concatemer (e.g., as shown in FIG. 7A, lower panel). In some embodiments, the oligonucleotide probes do not comprise a click functional group, and are therefore not ligated in a connecting step.

Exemplary first and second oligonucleotide probes can include CGTTCCTACCTTTCGTTCCTACC (SEQ ID NO: 9) and GCCCTAATTATTTTGCCCTAATTAT (SEQ ID NO: 10) in which CGTTCCTACC (SEQ ID NO: 11) is a first hybridizing region, GCCCTAATTAT (SEQ ID NO: 12) is a second hybridizing region adjacent to the first hybridizing region in the nucleic acid concatemer, and TTT is a linker.

FIG. 7B depicts another exemplary design of an oligonucleotide probe set provided herein. In some embodiments, stabilization of a nucleic acid relies upon one or more oligonucleotide probes (e.g., an oligonucleotide probe set). In some embodiments, the oligonucleotide probes are the same. In some embodiments, the oligonucleotide probes are different. In some embodiments, the first oligonucleotide probe comprises a first hybridizing region complementary to the oligonucleotide probe recognition site or portion thereof (e.g., a first recognition sequence or a second recognition sequence) on the nucleic acid concatemer, and a second hybridizing region complementary to the oligonucleotide probe recognition site or portion thereof (e.g., a first recognition sequence or a second recognition sequence) on the nucleic acid concatemer, wherein the hybridizing regions are separated by a linker region. In some embodiments, the second oligonucleotide probe comprises a first hybridizing region complementary to the oligonucleotide probe recognition site or potion thereof (e.g., a first recognition sequence or a second recognition sequence) on the nucleic acid concatemer, and a second hybridizing region complementary to the oligonucleotide probe recognition site or potion thereof (e.g., a first recognition sequence or a second recognition sequence) on the nucleic acid concatemer, wherein the third and fourth hybridizing regions are separated by a linker region. In some embodiments, the hybridizing regions do not hybridize to each other. In some embodiments, the linker region does not hybridize to the nucleic acid concatemer. In some embodiments, the linker region is between about 0 and about 200 nucleotides in length. In some embodiments, the linker regions are the same. In some embodiments the linker regions are different. In some embodiments, the linker regions have sequences that are complementary to one another and may hybridize.

Exemplary first and second oligonucleotide probes can include CGTTCCTACCTCACCGCTTCCGTTCCTACC (SEQ ID NO: 13) and GCCCTAATTATGAAGCGGTGAGCCCTAATTAT (SEQ ID NO: 14) in which CGTTCCTACC (SEQ ID NO: 11) is a first hybridizing region, GCCCTAATTAT (SEQ ID NO: 12) is a second hybridizing region adjacent to the first hybridizing region in the nucleic acid concatemer, and TCACCGCTTC (SEQ ID NO: 15) and GAAGCGGTGA (SEQ ID NO: 16) are complementary linker sequences.

In some embodiments, the oligonucleotide probe is covalently labeled with a detectable label via an optional linker. In some embodiments, the oligonucleotide probes is not covalently labeled with a detectable label via an optional linker. In some embodiments, the first hybridizing region on the first oligonucleotide probe and the second hybridizing region on the first oligonucleotide probe have the same sequence, and the first hybridizing region on the second oligonucleotide probe and the second hybridizing region on the second oligonucleotide probe have the same sequence. In some embodiments, the hybridizing regions of the oligonucleotide probes are all different. In some embodiments, the oligonucleotide probes do not comprise a click functional group, and are therefore not ligated in a connecting step.

In some embodiments, the second oligonucleotide probe is oriented so that the 5′ and 3′ ends of the probe are adjacent to the hybridizing regions of the first oligonucleotide probe. In some embodiments, the linker regions of the first oligonucleotide probe and the second oligonucleotide probe are not adjacent to one another, and do not hybridize to one another. In some embodiments, the second oligonucleotide probe is oriented so that the linker region is adjacent to the linker region of the first oligonucleotide probe. In some embodiments, the adjacent linker regions of the first and second oligonucleotide probe are complementary and may hybridize to one another.

### (ii) Click Functional Groups

The oligonucleotide probes provided herein may comprise functional groups. In some embodiments, the one or more oligonucleotide probes comprise functional groups at the termini of the probe. In some embodiments, the oligonucleotide probe comprises a functional group at the 3′ end of the probe. In some embodiments, the oligonucleotide probe comprises a functional group at the 5′ end of the probe. In some embodiments, the functional group comprises a click functional group. In some embodiments, the 5′ and/or 3′ ends of the oligonucleotide probe comprises click functional groups.

In some embodiments, the click functional groups are selected from the group consisting of 3′-azido / 5′-alkynyl, 3′-alkynyl / 5′-azido, 3′-azido / 5′-cyclooctynyl, 3′-cyclooctynyl / 5′-azido, 3′-tetrazine / 5′-dienophile, 3′-dienophile / 5′-tetrazine, 3′-thiol / 5′-alkynyl, 3′-alkynyl / 5′-thiol, 3′-cyano / 5′-1,2-amino thiol, 3′-1,2-amino thiol / 5′- cyano, 3′-nitrone / 5′-cyclooctynyl, and 3′-cyclooctynyl / 5′-nitrone. In some embodiments, the oligonucleotide probe comprises a 5′ azide and a 3′ alkyne. In some embodiments, the oligonucleotides probe comprises a 3′ alkyne and a 5′ azide. The specific click reactions are further described below.

### B. Oligonucleotide Probe Hybridization and Ligation

### (i) Oligonucleotide Probe Hybridization

In some aspects of the methods provided herein, one or more oligonucleotide probes hybridize (e.g., via a hybridizing region) to an oligonucleotide probe recognition site on a nucleic acid concatemer (e.g., RCP) in a biological sample. The ends of the one or more oligonucleotide probes hybridized to different copies of the oligonucleotide probe recognition site in the nucleic acid concatemer may be connected (e.g., ligated). In some embodiments, the connecting of the ends of the oligonucleotide probe stabilizes and/or compacts the nucleic acid concatemer in the biological sample.

In some embodiments, the biological sample comprising a nucleic acid concatemer is contacted with one or more oligonucleotide probes, each comprising at least one hybridizing region. The oligonucleotide probes may be any of the oligonucleotide probes described herein. In some embodiments, the nucleic acid concatemer comprises multiple copies of an oligonucleotide probe recognition site. In some embodiments, the one or more oligonucleotide probes hybridize with the oligonucleotide probe recognition site, or a portion thereof, in the nucleic acid concatemer. In some embodiments, the biological sample is contacted with a plurality of oligonucleotide probes.

In some embodiments, the plurality of oligonucleotide probes hybridize with multiple copies of the oligonucleotide probe recognition site, or a portion thereof. In some embodiments, the hybridizing regions of a first oligonucleotide probe of a plurality of oligonucleotide probes hybridizes with two different copies of the oligonucleotide probe recognition site. In some embodiments, one hybridizing region of an oligonucleotide probe may hybridize with the oligonucleotide probe recognition site, or portion thereof, while a second hybridizing region of the same oligonucleotide probe does not hybridize with the oligonucleotide probe recognition site. In some embodiments, each hybridizing region of an oligonucleotide probe hybridizes to a copy of the oligonucleotide probe recognition site.

In some embodiments, the hybridizing regions (e.g., a first hybridizing region and a second hybridizing region) of a first oligonucleotide probe hybridize to a first copy and a second copy of the oligonucleotide probe recognition site. In some embodiments, the hybridizing regions (e.g., a first hybridizing region and a second hybridizing region) of a first oligonucleotide probe are hybridized to a first copy and a second copy of the oligonucleotide probe recognition site.

In some embodiments, the hybridizing regions (e.g., a first hybridizing region and a second hybridizing region) of a first oligonucleotide probe are hybridized to a first copy and a second copy of the oligonucleotide probe recognition site. In some embodiments, the hybridizing regions of a second oligonucleotide probe are hybridized to a first copy and a second copy of the oligonucleotide probe recognition site. In some embodiments wherein the hybridizing regions of the first oligonucleotide probe are hybridized to a first copy and a second copy of the oligonucleotide probe recognition site, the hybridizing regions of a second oligonucleotide probe are hybridized to a first copy and a third copy of the oligonucleotide probe recognition site. In some embodiments wherein the hybridizing regions of the first oligonucleotide probe are hybridized to a first copy and a second copy of the oligonucleotide probe recognition site, the hybridizing regions of a second oligonucleotide probe are hybridized to a second copy and a third copy of the oligonucleotide probe recognition site.

In some embodiments, the hybridizing regions of each oligonucleotide probe are identical. In some embodiments, the first hybridizing region and the second hybridizing region of an oligonucleotide probe are identical.

In some embodiments, the nucleic acid concatemer in the biological sample is contacted with a plurality of splints (e.g., one or more splints). In some embodiments, the nucleic acid concatemer is contacted with the plurality of splints prior to being contacted with the one or more oligonucleotide probes. In some embodiments, the nucleic acid concatemer is contact with the plurality of splints after being contacted with the one or more oligonucleotide probes. In some embodiments, the splint is separate from the one or more oligonucleotide probes and the nucleic acid concatemer. In some embodiments, the splint does not hybridize to the nucleic acid concatemer. In some embodiments, the splint comprises a sequence that hybridizes to the nucleic acid concatemer. In some embodiments, the sequence that hybridizes to the nucleic acid concatemer is on the end of the splint (e.g., the 5′ or 3′ termini). In some embodiments, the sequence that hybridizes to the nucleic acid concatemer comprises between about 5 and about 20 nucleotides in length. In some embodiments, the sequence of the splint that hybridizes to the nucleic acid concatemer provides increased stability for the splint.

In one aspect, disclosed herein is a splint used to facilitate the ligation of one or more oligonucleotide probes hybridized to oligonucleotide probe recognition regions on a nucleic acid concatemer. In some embodiments, the splint comprises a first region immediately adjacent to a second region, where the first and second regions of the splint hybridize to the overhangs of one or more oligonucleotide probes disclosed herein. In some embodiments, the splint comprises a first region that is not immediately adjacent to a second region, e.g., there is one or more nucleotides between the first and second regions, and upon splint hybridization to the overhangs, the ends of the overhangs are separated by a gap and require gap filling prior to nick ligation. In some embodiments, the probe recognition regions are complementary to the a portion of the overhangs (e.g., a first and a second splint recognition region) of the oligonucleotide probes. The splint is separate from one or more oligonucleotide probes and the nucleic acid concatemer. In some embodiments, the splint may not hybridize to the nucleic acid concatemer. In some embodiments, the splint may comprise an additional sequence that hybridizes to the nucleic acid concatemer (e.g., at the 5′ end and/or the 3′ end of the splint), that is separate from the probe hybridizing regions. In some embodiments, the splint is covalently labeled with a detectable label via an optional linker. In some embodiments, the splint is not covalently labeled with a detectable label.

In some embodiments, the splint is a nucleic acid splint. In some embodiments, the splint is between about 5 and about 50 nucleotides in length, such as any of between about 5 and about 20, about 15 and about 30, about 25 and about 40, and about 30 and about 50 nucleotides in length. In some embodiments, the splint is about 10, about 15, about 20, or about 25 nucleotides in length.

In some embodiments, the splint is labeled with a detectable label (e.g., a fluorophore). In some embodiments, the splint is covalently labeled with a detectable label. In some embodiments, the splint is covalently labeled with a detectable label via a linker. Any suitable linker is acceptable to be used to covalently label the splint. In some embodiments, the detectable label is on the 5′ or 3′ end of the splint. In some embodiments, the splint is labeled with a detectable label on the 5′ end, the 3′ end, or both the 5′ and 3′ ends.

In some embodiments, the oligonucleotide probes do not hybridize to the splint. In some embodiments, the ends of the oligonucleotide probes do not hybridize to the splint. In some embodiments, the oligonucleotide probes hybridizes to the splint. In some embodiments, the splints hybridize with one or more oligonucleotide probes that are not hybridized with oligonucleotide probe recognition sites of the nucleic acid concatemer. In some embodiments, the splints hybridize with one or more oligonucleotide probes that are hybridized with oligonucleotide probe recognition sites of the nucleic acid concatemer. In some embodiments, a portion of the plurality of splints hybridize with one or more oligonucleotide probes that are hybridized with the oligonucleotide probe recognition site.

In some embodiments, the oligonucleotide probe hybridizes with one or more splints, such as hybridizing with one splint, two splints, three splints, or four splints. In some embodiments, the splint hybridizes to the overhang of the oligonucleotide probe. In some embodiments, the splint hybridizes to each overhang of the oligonucleotide probe. In some embodiments, the portion of the overhang (e.g., the splint recognition region) of the oligonucleotide probe are hybridized to the splint. In some embodiments, the splint recognition region of an overhang of an oligonucleotide probe comprises a sequence that specifically hybridizes to splint, or a portion thereof. In some embodiments, the splint hybridizes with the overhang (e.g., the splint recognition region) of one or more oligonucleotide probes. In some embodiments, the splint hybridizes with two oligonucleotide probes simultaneously. In some embodiments, two splints hybridize with two oligonucleotides probes simultaneously.

### (ii) Oligonucleotide Probe Connection

In some embodiments, the method further comprises connecting the ends of one or more oligonucleotide probes. In some embodiments, the oligonucleotide probe hybridization step (to a nucleic acid concatemer) and the step of connecting the ends of the oligonucleotide probes may happen in a single-pot reaction, or in two separate reactions in series. In some embodiments, the oligonucleotide probe hybridization step and the probe end connecting step may happen in parallel and/or subsequent to the generation of the concatemer (e.g., via RCA). In some embodiments, the oligonucleotide probe hybridization step, the probe end connecting step, and the generation of the concatemer (e.g., via RCA) may occur in any suitable order and in any suitable combination. For instance, the oligonucleotide probes can hybridize to a concatemer as it is being generated in situ, the ends of the oligonucleotide probes are connected, and then the concatemer can be further elongated.

In some embodiments, the connecting of the ends of the one or more oligonucleotide probes occurs while the probes are hybridized to the oligonucleotide probe recognition sites of the nucleic acid concatemer, or a portion thereof. In some embodiments, the connecting of the ends comprises connecting the overhangs of one or more oligonucleotide probes hybridized to the nucleic acid concatemer. In some embodiments, the connecting of the ends comprises connecting the functional groups at the 5′ and 3′ ends of the overhangs of one or more oligonucleotide probes hybridized to the nucleic acid concatemer. In some embodiments, the connecting of the ends of the overhangs of the one or more oligonucleotide probes is mediated by a splint.

In some embodiments, the connecting step does not comprise covalently connecting the overhangs. In some embodiments, the overhangs are covalently connected. In some embodiments, the connecting of the ends of one or more oligonucleotide probes comprises ligating the ends of one or more oligonucleotide probes hybridized to the oligonucleotide probe recognition sites of the nucleic acid concatemer, or a portion thereof.

In some embodiments, one or more oligonucleotide probes disclosed herein can be used as rolony stabilizing components (RSC). In some embodiments, a linkage between two or more RSCs is formed, such as by hydrogen bonding by a bridging oligonucleotide, and/or formation of chemical linkages between two or more RSCs, such as by enzymatic or chemical ligation. Enzymatic ligation may be by a DNA ligase, e.g., T4 DNA ligase. Chemical ligation may be by click reaction, such as by inclusion of an azide and alkyne on the RSC. The enzymatic and chemical ligations may be “splinted”, such as by hybridization of an accessory component complementary to two or more of the non-concatemer-hybridizing sequence domains of the RSC(s). The RSCs can be hybridized to the concatemers at saturating concentration, e.g., in significant molar excess to the oligonucleotide probe recognition sites, such that a substantial fraction of the oligonucleotide probe recognition sites are hybridized to RSCs, while avoiding the kinetic traps.

In some embodiments, the ligation comprises the joining of a 5′ phosphate and a 3′ hydroxyl of one or more oligonucleotide probes. In some embodiments, the ligation comprises ligating a 5′ and 3′ functional group of one or more oligonucleotide probes. In some embodiments, the functional group is a click functional group (e.g., any of the click functional groups described above). In some embodiments, the oligonucleotide probes comprise a 5′ azide and a 3′ alkyne. In some embodiments, the ligation is performed using any suitable technique.

In some embodiments, the ligation involves chemical ligation. In some embodiments, the ligation involves template dependent ligation (e.g., with a splint). In some embodiments, the ligation involves template independent ligation. In some embodiments, the ligation involves enzymatic ligation. In some embodiments, the ligation involves template dependent chemical ligation (e.g., via click chemistry).

In some embodiments, ligation of the one or more hybridized oligonucleotide probes produces a circular product hybridized to the nucleic acid concatemer with a melting temperature higher than that of unligated hybridized oligonucleotide probes. Thus, in some aspects, ligation stabilizes the nucleic acid concatemer containing the ligated oligonucleotide probes prior to subsequent analysis. In some embodiments, the ligation of the one or more hybridized oligonucleotide probes produces a circular product hybridized to the nucleic acid concatemer that is compacted compared to that of unligated hybridized oligonucleotide probes. Thus, in some aspects, ligation compacts the nucleic acid concatemer containing the ligated oligonucleotide probes prior to subsequent analysis.

In some aspects, a high fidelity ligase, such as a thermostable DNA ligase (e.g., a Taq DNA ligase), is used to ligate the ends of one or more oligonucleotide probes. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two nucleic acid sequences of one or more oligonucleotide probes, that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the ends of the one or more oligonucleotide probes (see, e.g., U.S. Pat. No. 7,264,929, the entire contents of which are incorporated herein by reference). A wide variety of different methods can be used for proximity ligating one or more oligonucleotide probes, including (but not limited to) “sticky-end” and “blunt-end” ligations. Additionally, single-stranded ligation can be used to perform proximity ligation on one or more oligonucleotide probes. Sticky-end proximity ligations involve the hybridization of complementary single-stranded sequences between the one or more oligonucleotide probes to be joined, prior to the ligation event itself. Blunt-end proximity ligations generally do not include hybridization of complementary regions from each end of the one or more oligonucleotide probes, because both molecules lack a single-stranded overhang at the site of ligation.

In some embodiments, the one or more oligonucleotide probes connected via their click functional groups using an enzyme-free click reaction (that is, without use of a polymerase or a ligase for conjugation). In some embodiments, the connecting step comprises ligating the ends (e.g., the ends of the overhangs) of one oligonucleotide probe. In some embodiments, the connecting step comprises ligating the ends (e.g., the ends of the overhangs) of two oligonucleotide probes. Any suitable click chemistry is contemplated for use with the compositions, systems, and methods described herein, for example see, e.g., Gartner and Liu (2001), Journal of the American Chemical Society 123(28): 6961-6963; Seckute et al. (2013), Nucleic Acids Research 41(15): e148; and Patterson et al. (2014), ACS Chem. Biol. 9(3): 592-605, all of which are incorporated by reference herein in their entirety. In some instances the click functional groups described herein comprise at one µM concentration per click functional group, a click reaction with a k₂ rate (M⁶s⁻¹) and associated half-life of 10⁵ and 10 seconds, 10⁴ and 1.7 minutes, 10³ and 17 minutes, 10² and 2.8 hours, 10 and 1.2 days, 1 and 12 days, 10⁻¹ and four months, or 10⁻² and three years.

In some embodiments, the click reaction is a template-independent reaction. Template-independent click reactions are known (see, e.g., Xiong and Seela (2011), J. Org. Chem. 76(14): 5584-5597, incorporated by reference herein in its entirety). In some embodiments, the click reaction is a nucleophilic addition reaction. In some embodiments, the click reaction is a cyclopropane-tetrazine reaction. In some embodiments, the click reaction is a strain-promoted azide-alkyne cycloaddition (SPAAC) reaction. In some embodiments, the click reaction is an alkyne hydrothiolation reaction. In some embodiments, the click reaction is an alkene hydrothiolation reaction. In some embodiments, the click reaction is a strain-promoted alkyne-nitrone cycloaddition (SPANC) reaction. In some embodiments, the click reaction is an inverse electron-demand Diels-Alder (IED-DA) reaction. In some embodiments, the click reaction is a cyanobenzothiazole condensation reaction. In some embodiments, the click reaction is an aldehyde/ketone condensation reaction. In some embodiments, the click reaction is a Cu(I)-catalyzed azide-alkyne cycloaddition (CuAAC) reaction.

In some embodiments, the click reaction is a template-dependent reaction or template-directed reaction. In some embodiments, the template-dependent reaction or template-directed reaction is a splint mediated reaction. In some embodiments, the splint is hybridized to the overhangs (e.g., the splint recognition regions of the overhangs) or one or more oligonucleotide probes. In some embodiments, the splint-mediated reaction is sensitive to base pair mismatches such that reaction rate is significantly higher for matched vs. unmatched splints. In some embodiments, the click reaction is a nucleophilic addition template-dependent reaction. In some embodiments, the click reaction is a cyclopropane-tetrazine template-dependent reaction.

In some embodiments, a first oligonucleotide probe is hybridized to a first copy of the oligonucleotide probe recognition site on the nucleic acid concatemer, and a second oligonucleotide probe is hybridized to a second copy of the oligonucleotide probe recognition site on the nucleic acid concatemer. In some embodiments, upon hybridization to the nucleic acid concatmener, the overhang regions of the individual probes are oriented so that the terminal functional groups are adjacent to one another. In some embodiments, the functional groups at the ends of the overhangs are covalently connected via a connecting step. In some embodiments, the connecting step comprises ligating the ends of the overhangs using enzymatic ligation. In some embodiments, the connecting step comprises ligating the ends of the overhangs using chemical ligation. In some embodiments, the connecting step comprises ligating the ends of the overhangs using template-dependent ligation (e.g., splint-dependent ligation). In some embodiments, the connecting step comprises ligating the ends of the overhangs using template-independent ligation. In some embodiments, the splint hybridizes to the overhang regions of the oligonucleotide probes (e.g., at the splint recognition regions) to facilitate ligation. In some embodiments, first oligonucleotide probe comprises a 5′ azide and a 5′ sequence that hybridizes to a splint. In some embodiments, the second oligonucleotide probe comprises a 3′ alkyne and a 3′ sequence that hybridizes to the splint. In some embodiments, upon hybridization of the first and second oligonucleotide probes to the splint, the 5′ azide and 3′ alkyne are in proximity for click reaction.

In some embodiments, a first copy of the oligonucleotide probe hybridizes to a first copy of the oligonucleotide probe recognition site on the nucleic acid concatemer at the probe’s hybridizing region, and a second copy of the oligonucleotide probe hybridizes to a second copy of the oligonucleotide probe recognition site on the nucleic acid concatemer at the probe’s hybridizing region. In some embodiments, upon hybridization of the probes to the nucleic acid concatemer, the overhang regions of the individual probes are oriented so that the 5′ end of the first oligonucleotide probe is adjacent to the 3′ end of the second oligonucleotide probe and the 3′ end of the first oligonucleotide probe is adjacent to the 5′ end of the second oligonucleotide probe. In some embodiments, the functional groups at the ends of the overhangs are covalently connected via a connecting step. In some embodiments, the connecting step comprises ligating the ends of the overhangs using enzymatic ligation. In some embodiments, the connecting step comprises ligating the ends of the overhangs using chemical ligation. In some embodiments, the connecting step comprises ligating the ends of the overhangs using template-dependent ligation. In some embodiments, the connecting step comprises ligating the ends of the overhangs using template-independent ligation. In some embodiments, the splint hybridizes to the overhang regions of the oligonucleotide probes at the splint recognition regions to facilitate ligation.

In some embodiments, the first oligonucleotide probe comprises a 5′ azide and a 5′ sequence that hybridizes to a first splint. In some embodiments, the first oligonucleotide probe further comprises a 3′ alkyne and a 3′ sequence that hybridizes to a second splint. In some embodiments, the second oligonucleotide probe comprises (a 3′ alkyne and a 3′ sequence that hybridizes to the first splint. In some embodiments, the second oligonucleotide probe further comprises a 5′ azide and a 5′ sequence that hybridizes to the second splint. In some embodiments, upon hybridization of the first and second oligonucleotide probes to the first and second splints, the 5′ azide of the first oligonucleotide and the 3′ alkyne of the second oligonucleotide are in proximity for click reaction, and the 3′ alkyne of the first oligonucleotide and the 5′ azide of the second oligonucleotide are in proximity for click reaction.

In some embodiments, the first hybridizing region of a single oligonucleotide probe hybridizes to a first copy of the oligonucleotide probe recognition site on the nucleic acid concatemer and the second hybridizing region of the same oligonucleotide probe hybridizes to a second copy of the oligonucleotide probe recognition site on the nucleic acid concatemer. In some embodiments, upon hybridization to the nucleic acid concatemer, the overhang regions of the oligonucleotide probes are oriented so the terminal functional groups are adjacent to one another. In some embodiments, the functional groups at the ends of the overhangs are covalently connected via a connecting step. In some embodiments, the connecting step comprises ligating the ends of the overhangs using enzymatic ligation. In some embodiments, the connecting step comprises ligating the ends of the overhangs using chemical ligation. In some embodiments, the connecting step comprises ligating the ends of the overhangs using template-dependent ligation. In some embodiments, the connecting step comprises ligating the ends of the overhangs using template-independent ligation. In some embodiments, the splint hybridizes to the overhang regions of the oligonucleotide probes at the splint recognition regions to facilitate ligation.

In some embodiments, in the connecting step, the ends of an oligonucleotide probe are connected, wherein the oligonucleotide probe comprises hybridizing regions that hybridize to different copies of the oligonucleotide probe recognition site. In some embodiments, the oligonucleotide probe further comprises a linker between the hybridizing regions that does not hybridize to the nucleic acid concatemer. In some embodiments, the linker is between about 0 and about 200 nucleotides in length. In some embodiments, the oligonucleotide probe comprises a 5′ azide and a 3′ alkyne, and a 5′ sequence and a 3′ sequence that hybridize to a splint, and upon hybridization to the splint, the 5′ azide and 3′ alkyne are in proximity for click reaction.

In some embodiments, the first hybridizing region of an oligonucleotide probe is hybridized to a first copy of the oligonucleotide probe recognition site on the nucleic acid concatemer, and the second hybridizing region of the same oligonucleotide probe is hybridized to a second copy of the oligonucleotide probe recognition site on the nucleic acid concatemer. In some embodiments, the ends of the oligonucleotide probe do not comprise a functional group. In some embodiments, the ends of the oligonucleotide probe are not ligated.

In some embodiments, each oligonucleotide probe recognition site on the nucleic acid concatemers comprises a first recognition sequence adjacent to a second recognition sequence. In some embodiments, the first and second recognition sequences are identical or different in sequence. In some embodiments, the first and second recognition sequences are identical or different in length. In some embodiments, the first hybridizing region of the first oligonucleotide probe, hybridizes to a first copy of the first oligonucleotide probe recognition site on the nucleic acid concatemer. In some embodiments, the second hybridizing region of the first oligonucleotide probe hybridizes to a second copy of the first oligonucleotide probe recognition site. In some embodiments, the first hybridizing region of the second oligonucleotide probe, hybridizes to a first copy of the second oligonucleotide probe recognition site on the nucleic acid concatemer. In some embodiments, the second hybridizing region of the second oligonucleotide probe hybridizes to a second copy of the second oligonucleotide probe recognition site on the nucleic acid concatemer. In some embodiments, the 5′ and 3′ ends of the second oligonucleotide probe are adjacent to the linker region of the first oligonucleotide probe. In some embodiments, the linker regions of the oligonucleotide probes do not hybridize to one another.

In some embodiments, each oligonucleotide probe recognition site on the nucleic acid concatemers comprises a first recognition sequence adjacent to a second recognition sequence. In some embodiments, the first and second recognition sequences are identical or different in sequence. In some embodiments, the first and second recognition sequences are identical or different in length. In some embodiments, the first hybridizing region of the first oligonucleotide probe hybridizes to a first copy of the first oligonucleotide probe recognition site on the nucleic acid concatemer. In some embodiments, the second hybridizing region of the first oligonucleotide probe hybridizes to a second copy of the first oligonucleotide probe recognition site. In some embodiments, the first hybridizing region of the second oligonucleotide probe hybridizes to a first copy of the second oligonucleotide probe recognition site on the nucleic acid concatemer. In some embodiments, the second hybridizing region of the second oligonucleotide probe hybridizes to a second copy of the second oligonucleotide probe recognition site on the nucleic acid concatemer. In some embodiments, the linker region of the second oligonucleotide probe is adjacent to the linker region of the first oligonucleotide probe. In some embodiments, the linker regions of the oligonucleotide probes hybridize to one another.

### C. Hydrogel Embedding

In some aspects, the methods provided herein further comprise embedding and/or immobilizing a sample (e.g., a biological sample) in a hydrogel matrix. In some embodiments, the nucleic acid concatemer (e.g., RCPs) generated by any of the methods described herein are embedded in a hydrogel matrix. Methods for embedding a sample in a matrix have been described in, e.g., US2016/0024555, US2019/0276881, US2020/0071751, WO2020/0076976, WO2020/0076979, and WO2020/0096687, which are incorporated herein by reference in their entirety. In some embodiments, the hydrogel comprises a three dimensional matrix. For example, the matrix may be a three dimensional nucleic acid-containing polymer. In some embodiments, the polymer is a gel or a hydrogel. In some embodiments, the nucleic acids described herein (e.g., oligonucleotide probes) may be bound a hydrogel matrix to stabilize a nucleic acid concatemer.

In some embodiments, the matrix may encapsulate a biological sample, such as a tissue sample. In some embodiments, the sample is embedded or immobilized, partially or completely, in the matrix. In some embodiments, the matrix comprises polyacrylamide, cellulose, alginate, polyamide, cross-linked agarose, cross-linked dextran or cross-linked polyethylene glycol. In some embodiments, the matrix comprises any suitable polymerizing and/or crosslinking material. In some embodiments, polymerization initiators and/or crosslinkers may be present. In some embodiments, the matrix is porous, such that exogenous reagents and/or oligonucleotide probes described herein may be introduced to the matrix.

In some aspects, the oligonucleotide probe bound to the three dimensional matrix comprises naturally or non-naturally occurring nucleic acids (e.g., synthetic or modified nucleic acids). In some embodiments, the oligonucleotide probe is modified to incorporate a functional moiety for attachment to the matrix (such as, by covalent cross-linking or non-covalent binding). In some embodiments, the functional moiety is bound to the oligonucleotide probe at the 3′ end of the probe. In some embodiments, the functional moiety is bound to the oligonucleotide probe at the 5′ end of the probe. In some embodiments, the functional moiety is cross-linked to a modified nucleic acid (e.g., dNTp or dUTP) on the oligonucleotide probe. In some embodiments, the cross-linker reactive group is any suitable reactive group.

In some embodiments, the matrix is modified to incorporate a functional moiety for attachment to the oligonucleotide probe. In some embodiments, the functional moiety of the oligonucleotide probe is attached (e.g., by covalent cross-linking or non-covalent binding) to the matrix via the functional group of the matrix. In some embodiments, the cross-linkers further include a spacer for attachment to the matrix. In some embodiments, the spacer comprises polyethylene glycol, carbon spacers, and/or photo-cleavable spacers.

The functional moiety of the oligonucleotide probe and/or the matrix may comprise a ligand. In some embodiments, the ligand reacts with a cross-linker or another ligand (e.g., the ligand of the oligonucleotide probe reacts with the ligand of the matrix). In some embodiments, the functional moiety is any of an amine, acrydite, alkyne, biotin, azide, and thiol. In some embodiments, the method comprises embedding an oligonucleotide probe comprising a functional moiety described herein to the matrix. In some embodiments, the functional moiety of the matrix is an alkyne, and the functional moiety of the oligonucleotide probe is an azide.

In some embodiments, a first oligonucleotide probe is hybridized to a first copy of the oligonucleotide probe recognition site on the nucleic acid concatemer. In some embodiments, the functional group at the end of overhang of the oligonucleotide probe is covalently connected via a connecting step to the hydrogel matrix in which the nucleic acid concatemer is embedded. In some embodiments, the functional group at the end of the overhang of the oligonucleotide probe is a 5′ azide, and the functional group attached to the hydrogel matrix is a 3′ alkyne. In some embodiments, the 5′ azide on the oligonucleotide probe and the 3′ alkyne attached to the hydrogel matrix are ligated through click chemistry.

In some embodiments, a copy of the oligonucleotide probe is hybridized to a first copy of the oligonucleotide probe recognition site on the nucleic acid concatemer. In some embodiments, the functional group at the end of overhang of the oligonucleotide probe is covalently connected via a connecting step to the hydrogel matrix in which the nucleic acid concatemer is embedded. In some embodiments, the functional group at the end of the overhang of the oligonucleotide probe is a 5′ azide, and the functional group attached to the hydrogel matrix is a 3′ alkyne. In some embodiments, the 5′ azide on the oligonucleotide probe and the 3′ alkyne attached to the hydrogel matrix are ligated through click chemistry.

In some embodiments, a first copy of the oligonucleotide probe hybridizes to a first copy of the oligonucleotide probe recognition site of the nucleic acid concatemer at the probe’s hybridizing region. In some embodiments, a functional group on the end of an overhang region on the first oligonucleotide probe is ligated to a functional group on the end of an overhang region on a second copy of the oligonucleotide probe during the connecting step. In some embodiments, a first splint hybridizes to the splint recognition regions of the first and second oligonucleotide probes’ overhang regions to facilitate ligation. In some embodiments, the hybridizing region of the second oligonucleotide probe does not hybridize to the nucleic acid concatemer. In some embodiments, the functional group on the second overhang region of the second oligonucleotide probe is ligated to a functional group on the end of an overhang region on a third copy of the oligonucleotide probe during the connecting step. In some embodiments, a second splint hybridizes to the splint recognition regions of the second and third oligonucleotide probes to facilitate ligation. In some embodiments, the hybridizing region of the third oligonucleotide probe hybridizes to a second copy of the oligonucleotide probe recognition site of the nucleic acid concatemer at the probe’s hybridizing region. In some embodiments, the second functional group on the second overhang region of the oligonucleotide probe is ligated to the hydrogel matrix in which the nucleic acid concatemer is embedded.

In some embodiments, the first hybridizing region of a first copy of the oligonucleotide probe is hybridized to a first copy of the oligonucleotide probe recognition site on the nucleic acid concatemer. In some embodiments, the functional group on the first overhang region of the oligonucleotide probe is ligated to the hydrogel matrix in which the nucleic acid concatemer is embedded. In this embodiment, the second hybridizing region of the first oligonucleotide probe is not hybridized to the nucleic acid concatemer. In some embodiments, a functional group on the end of an overhang region on the first oligonucleotide probe is ligated to a functional group on the end of an overhang region on a second copy of the oligonucleotide probe during the connecting step. In some embodiments, a first splint hybridizes to the splint recognition regions of the first and second oligonucleotide probes to facilitate ligation. In some embodiments, the first hybridizing region of the second oligonucleotide probe is not hybridized to the nucleic acid concatemer. In some embodiments, the second hybridizing region of the second oligonucleotide probe is hybridized to a second copy of the oligonucleotide probe recognition site on the nucleic acid concatemer.

### V. Detection and Analysis

In some aspects, the provided methods involve analyzing, e.g., detecting or determining, one or more sequences present in the target nucleic acid and/or in the nucleic acid concatemers described herein. In some embodiments, the detecting comprises hybridizing one or more detectably labeled probes to the nucleic acid concatemer, or via hybridization to adaptor probes that hybridize to the nucleic acid concatemer). In some embodiments, the analysis comprises determining the sequence of all or a portion of the nucleic acid concatemer (e.g., a barcode sequence or a complement thereof), wherein the sequence is indicative of a sequence of the target nucleic acid.

In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential probe hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise sequencing all or a portion of the nucleic acid concatemer, such as one or more barcode sequences present in the nucleic acid concatemer. In some embodiments, the sequence of the nucleic acid concatemer, or barcode thereof, is indicative of a sequence of the target nucleic acid to which the nucleic acid concatemer is hybridized. In some embodiments, the analysis and/or sequence determination comprises sequencing all or a portion of the nucleic acid concatemer and/or in situ hybridization to the nucleic acid concatemer. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US2017/0009278, which is incorporated herein by reference, for exemplary probes and HCR reaction components. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product/nucleic acid concatemer).

In some aspects, the provided methods comprise imaging the probe hybridized to the nucleic acid concatemer, for example, via binding of the secondary probe (e.g., a detection probe) and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a secondary probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more nucleic acid concatemer(s) described herein. In some embodiments, the methods involve incubating the detectable probe containing the detectable label with the sample, washing unbound detectable probe, and detecting the label, e.g., by imaging. In some embodiments, the nucleic acid concatemer(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227- 259 (1991), which are herein incorporated by reference in their entireties. In some embodiments, exemplary techniques and methods methodologies applicable to the provided embodiments comprise those described in, for example, US 4,757,141, US 5,151,507 and US 5,091,519, which are herein incorporated by reference in their entireties. In some embodiments, one or more fluorescent dyes are used as labels for labeled target sequences, for example, as described in US 5,188,934 (4,7-dichlorofluorescein dyes); US 5,366,860 (spectrally resolvable rhodamine dyes); US 5,847,162 (4,7- dichlororhodamine dyes); US 4,318,846 (ether-substituted fluorescein dyes); US 5,800,996 (energy transfer dyes); US 5,066,580 (xanthine dyes); and US 5,688,648 (energy transfer dyes). Labelling can also be carried out with quantum dots, as described in US 6,322,901, US 6,576,291, US 6,423,551, US 6,251,303, US 6,319,426, US 6,426,513, US 6,444,143, US 5,990,479, US 6,207,392, US 2002/0045045 and US 2003/0017264, which are herein incorporated by reference in their entireties. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, B ODIPY TMFL-14-dUTP, B ODIPY TMR-14-dUTP, B ODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein- 12-UTP, tetramethylrhodamine-6-UTP, TEXAS RED™-5- UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345, the entire contents of which are incorporated herein by reference).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, the entire contents of which are incorporated herein by reference).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a- digoxigenin, dinitrophenol (DNP)/a-DNP, 5-Carboxyfluorescein (FAM)/a-FAM.

In some embodiments, a nucleotide and/or an oligonucleotide sequence can be indirectly labeled, especially with a hapten that is then bound by a capture agent, e.g., as disclosed in US 5,344,757, US 5,702,888, US 5,354,657, US 5,198,537 and US 4,849,336, and PCT publication WO 1991/017160, which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image’s optical resolution, particularly in the sample depth direction, is much better than that of wide-field microscopes. However, as much of the light from sample fluorescence is blocked at the pinhole, this increased resolution is at the cost of decreased signal intensity - so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low- voltage electron microscopy (LVEM), scanning probe microscopy (SPM), atomic force microscopy (ATM), ballistic electron emission microscopy (BEEM), chemical force microscopy (CFM), conductive atomic force microscopy (C- AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/ microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, the entire contents of each of which are incorporated herein by reference. In addition, examples of methods and systems for performing in situ sequencing are described in US 2016/0024555, US 2019/0194709, and in US 10,138,509, US 10,494,662 and US 10,179,932, the entire contents of each of which are incorporated herein by reference. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121), the entire contents of each of which are incorporated herein by reference.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, US 7,057,026, US 2006/0240439, US 2006/0281109, US 2011/0059865, US 2005/0100900, US 9,217,178, US 2009/0118128, US 2012/0270305, US 2013/0260372, and US 2013/0079232, which are herein incorporated by reference in their entireties.

In some embodiments, sequence analysis of nucleic acids (e.g., nucleic acids such as probes or RCA products comprising barcode sequences) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference. In some embodiments, the methods provided herein can include analyzing the identifier sequences (e.g., analyte sequences or barcode sequences) by sequential hybridization and detection with a plurality of labeled probes (e.g., detection oligonucleotides).

In some embodiments, provided herein are methods for in situ analysis of analytes in a sample using sequential probe hybridization. In some aspects provided herein is a method for analyzing a biological sample, comprising: a) generating a rolling circle amplification product (RCP) of a circular or circularizable probe or probe set described herein, the RCP comprising an identifier sequence such as a barcode sequence or analyte sequence, wherein the identifier sequence is associated with an analyte of interest and is assigned a signal code sequence; b) contacting the biological sample with a first probe (e.g., an intermediate probe such as an L-probe) and a first detectably labeled probe to generate a first complex comprising the first probe hybridized to the RCP and the first detectably labeled probe hybridized to the first probe, wherein the first probe comprises (i) a recognition sequence (e.g., a target-binding sequence) complementary to the identifier sequence (e.g., barcode sequence or analyte sequence) and (ii) a first landing sequence (e.g., an overhang sequence), and wherein the first detectably labeled probe comprises a sequence complementary to the first landing sequence; c) detecting a first signal associated with the first detectably labeled probe, wherein the first signal corresponds to a first signal code in the signal code sequence; d) contacting the biological sample with a second probe (e.g., an intermediate probe such as L-probe) and a second detectably labeled probe to generate a second complex comprising the second probe hybridized to the RCP and the second detectably labeled probe hybridized to the second probe, wherein the second probe comprises (i) a recognition sequence (e.g., a target-binding sequence) complementary to the identifier sequence (e.g., barcode sequence or analyte sequence) and (ii) a second landing sequence (e.g., an overhang sequence), and wherein the second detectably labeled probe comprises a sequence complementary to the second landing sequence; and e) detecting a second signal associated with the second detectably labeled probe, wherein the second signal corresponds to a second signal code in the signal code sequence, wherein the signal code sequence comprising the first signal code and the second signal code is determined at a location in the biological sample, thereby decoding the identifier sequence (e.g., barcode sequence or analyte sequence) and identifying the analyte of interest at the location in the biological sample. In some embodiments, the detectable label of the first detectably labeled probe and the detectable label of the second detectably labeled probe are the same. In some embodiments, the detectable labels of the first detectably labeled probe and the second detectably labeled probe are different. In some embodiments, the first signal code and the second signal code are the same. In some embodiments, the first signal code and the second signal code are different.

In some embodiments, the first probe (e.g., a first intermediate probe such as a first L-probe), the second probe (e.g., a second intermediate probe such as a second L-probe), and one or more subsequent probes (e.g., subsequent intermediate probe such as subsequent L-probes) are contacted with the biological sample sequentially in a pre-determined sequence which corresponds to the signal code sequence assigned to the identifier sequence (e.g., barcode sequence or analyte sequence), wherein the one or more subsequent probes each comprises (i) a recognition sequence complementary to the identifier sequence (e.g., barcode sequence or analyte sequence) and (ii) an overhang sequence complementary to a detectably labeled probe of a pool (e.g., a universal pool across different cycles of probe hybridization) of detectably labeled probes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first detectably labeled probe and the second detectably labeled probe are in the pool of detectably labeled probes. A pool of detectably labeled probes may comprises at least two detectably labeled probes, and may be used for multiplexing analyses of two or more target analytes (e.g., target nucleic acids) in a biological sample. In some embodiments, the contacting in b) comprises contacting the biological sample with the universal pool of detectably labeled probes, and the contacting in d) comprises contacting the biological sample with the universal pool of detectably labeled probes. In some embodiments, the universal pool of detectably labeled probes used in the contacting in b) is the same as the universal pool of detectably labeled probes used in the contacting in d). In some embodiments, the universal pool comprises detectably labeled probes each having a detectable label corresponding to a different nucleic acid sequence for hybridization to a landing sequence (e.g., an overhang sequence) in a probe (e.g., an intermediate probe such as an L-probe). In some embodiments, the number of different detectably labeled probes in the universal pool is four.

In some embodiments, the one or more subsequent probes are contacted with the biological sample to determine signal codes in the signal code sequence until sufficient signal codes have been determined to decode the identifier sequence (e.g., barcode sequence or analyte sequence), thereby identifying the target analyte (e.g., target nucleic acid). In some embodiments, the method further comprises a step of removing the first probe and/or the first detectably labeled probe from the biological sample before contacting the sample with a subsequent probe and a detectably labeled probe hybridizing to the subsequent probe. In some embodiments, the method further comprises a step of removing the second probe and/or the second detectably labeled probe from the biological sample, before contacting the sample with a subsequent probe and a detectably labeled probe hybridizing to the subsequent probe.

In some embodiments, the method further comprises identifying multiple different target analytes present at locations (e.g., different locations) in the biological sample. In some embodiments, each different target analyte is assigned a different signal code sequence and is targeted by a circular or circularizable probe or probe set comprising a complement of a different barcode sequence of the plurality of barcode sequences. In some embodiments, the number of different probes (e.g., L-probes that have different recognition sequences that bind to the barcode sequences) in each pool of probes is greater than the number of different detectably labeled probes in the universal pool of detectably labeled probes. In some embodiments, the number of different detectably labeled probes in the universal pool is four. In some embodiments, the number of different probes in each pool of probes (e.g., L-probes) is about 10, about 20, about 30, about 40, about 50, about 100, about 200, about 500, about 1,000, or more. In some embodiments, the number of different recognition sequences (e.g., recognition sequences that bind to the barcode sequences) of probes in each pool of probes in at least about 10, such as at least any of about 20, 30, 40, 50, 100, 200, 500, 1,000, or more.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in US 5,599,675; US 5,750,341; US 6,969,488; US 6,172,218; US and 6,306,597, the entire contents of each of which are incorporated herein by reference.

In some embodiments, the barcodes of the detection probes are targeted by detectably labeled secondary probe oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; US 10,457,980 B2; US 2016/0369329 A1; WO 2018/026873 A1; US2020/0080139 A1; US20210017587 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in US 8,460,865, and in Gunderson et al., Genome Research 14:870-877 (2004), the entire contents of each of which are incorporated herein by reference.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, the entire contents of each of which are incorporated herein by reference.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VI. Compositions, Kits, and Systems

In some embodiments, disclosed herein is a composition that comprises a complex containing a target nucleic acid (e.g., a target nucleic amplification product, such as a rolling circle amplification product), an oligonucleotide probe, and an oligonucleotide probe recognition site of a nucleic acid concatemer, e.g., any of the target nucleic acids, oligonucleotide probes, and circular probes described in Section IV. In some embodiments, the complex further comprises a secondary probe (e.g., a detection probe), e.g., as described in Section V. In some embodiments, the end of one or more oligonucleotide probes hybridized to a nucleic acid concatemer are connected, such that the nucleic acid concatemer is stabilized and/or compacted.

In some embodiments, a kit provided herein comprses an oligonucleotide probe and a splint, wherein the oligonucleotide probe comprises (i) a hybridizing region that hybridizes to an oligonucleotide probe recognition site or portion thereof on a nucleic acid concatemer, and (ii) an overhang that does not hybridize to the nucleic acid concatemer, and the splint comprises a sequence that hybridizes to the overhang or a portion thereof of the oligonucleotide probe.

Also provided herein are kits, for example comprising one or more oligonucleotide probes, e.g., any described in Section IV, and instructions for performing the methods provided herein. In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Sections III and IV. In some embodiments, any or all of the oligonucleotides are DNA molecules. In some embodiments, the target nucleic acid is a messenger RNA molecule. In some embodiments, the target nucleic acid is a probe (e.g., a circularizable probe such as a padlock probe) or an amplification product thereof (e.g., a rolling circle amplification product, such as a nucleic acid concatemer). The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, and reagents for additional assays.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to compact nucleic acid concatemers via stabilization/compaction probes, to increase the resolution and stability of the nucleic acid concatemers in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids can be used. As indicated by standard references, a simple estimate of the Tₘ value may be calculated by the equation, Tₘ =81.5 + 0.41 (% G + C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5 × SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1 × SSPE, 0.1% SDS, 65° C.; 2) medium stringency: 0.2 × SSPE, 0.1% SDS, 50° C. (also referred to as moderate stringency); and 3) low stringency: 1.0 × SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5 × Denhardt’s solution, 5x SSPE, 0.2% SDS at 42° C., followed by washing in 0.2 × SSPE, 0.2% SDS, at 42° C. High stringency conditions can be provided, for example, by hybridization in 50% formamide, 5 × Denhardt’s solution, 5 × SSPE, 0.2% SDS at 42° C., followed by washing in 0.1 × SSPE, and 0.1% SDS at 65° C. Low stringency hybridization can refer to conditions equivalent to hybridization in 10% formamide, 5 × Denhardt’s solution, 6 × SSPE, 0.2% SDS at 22° C., followed by washing in lx SSPE, 0.2% SDS, at 37° C. Denhardt’s solution contains 1% Ficoll, 1% polyvinylpyrolidone, and 1% bovine serum albumin (BSA). 20 × SSPE (sodium chloride, sodium phosphate, ethylene diamide tetraacetic acid (EDTA)) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999), the entire contents of each of which are incorporated herein by reference.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and is not intended to limit the scope of the present disclosure.

### Example 1: Oligonucleotide Probes and Splints for the Stabilization and/or Compaction of a Rolling Circle Amplification Product

This example demonstrates a method for analyzing a biological sample using an oligonucleotide probe and a splint. In particular, this example demonstrates the compaction and stabilization of a rolling circle amplification (RCA) product (RCP) to reduce the size of the resulting RCP (e.g., nucleic acid concatemer) and lock it in place, in order to properly resolve densely packed RCP during downstream analyses while keeping the fluorescence intensity the same.

FIG. 9 shows an exemplary workflow for the stabilization and/or compaction of nucleic acid concatemers using the methods described herein. A biological sample (e.g., a processed or cleared biological sample, a tissue sample, a sample embedded in a hydrogel, etc.) is contacted with a primary probe, such as a circularizable probe or probe set (e.g., a padlock probe). The padlock probe can comprise a 5′ phosphate group, optionally a 3′ RNA base, and an oligonucleotide probe recognition site. The oligonucleotide probe recognition site or complement thereof is capable of hybridizing with an oligonucleotide probe (e.g., any of the stabilization and/or compaction oligonucleotide probes described herein). Padlock probes are hybridized to a target nucleic acid sequence, such as an mRNA or cDNA, in the tissue sample and are ligated to generate a closed circle from the padlock probe. The closed circle is then amplified by a DNA polymerase in a RCA reaction to generate a RCP comprising two or more oligonucleotide probe recognition sites.

Upon generation of the RCP comprising two or more oligonucleotide probe recognition sites, the biological sample is contacted with a plurality of oligonucleotide probes, e.g., as shown in FIG. 9. The oligonucleotide probes comprise at least one hybridizing region complementary to the oligonucleotide probe recognition site, or portion thereof, on the RCP, and are capable of hybridizing with the oligonucleotide probe recognition site, or portion thereof. The oligonucleotides may further comprise one or more overhangs. The overhangs do not hybridize with the RCP, and are not complementary to the oligonucleotide probe recognition site or portion thereof. The overhangs of the plurality of oligonucleotide probes may comprise one or more terminal functional groups (e.g., click functional groups) to be used to ligate the probe to itself, to one or more additional oligonucleotide probes, or to the hydrogel matrix. Optionally, the overhangs may comprise a splint recognition region that is capable of hybridizing with a splint.

The plurality of oligonucleotide probes, or a portion thereof, hybridize with the oligonucleotide probe recognition sites, or a portion thereof, on the RCP. Upon hybridization of the oligonucleotide probes, a plurality of splints are introduced to the RCP. The splints do not hybridize with the RCP, but are capable of hybridizing with the overhangs of the hybridized oligonucleotide probes via probe recognition regions (e.g., a first and a second probe recognition region). Specifically, the splints are capable of hybridizing with a splint recognition region on the overhang of the oligonucleotide probes. A first probe recognition region of the splint hybridizes with the overhang of an oligonucleotide probe, and a second probe recognition region of the same splint hybridizes with the overhang of a second oligonucleotide probe that is hybridized to a different copy of the oligonucleotide probe recognition site on the RCP.

The splints may be used to mediate the ligation of the overhangs of the hybridized oligonucleotide probes. In particular, the splints mediate the ligation of the click functional groups at the termini of the oligonucleotide probes by bringing the functional groups into close proximity. In some embodiments, the oligonucleotide probes comprise a 5′ azide and a 3′ alkyne that are capable of being ligated to a 3′ alkyne and 5′ azide, respectively, of a second oligonucleotide probe hybridized to a second copy of the oligonucleotide recognition site on the RCP.

The hybridization of the oligonucleotide probes to the nucleic acid concatemer followed by the subsequent ligation of the ends of the individual probes (e.g., via splint-mediated ligation) results in stabilization of the nucleic acid concatemer. Stabilization of the nucleic acid concatemer allows the concatemer to remain in the same configuration following rounds of in situ analysis and processing, such as in situ hybridization or sequencing. In some embodiments, the oligonucleotide probes also facilitate compaction of the nucleic acid concatemer in addition to stabilization. Thus, the oligonucleotide probes will not be stripped by reagents used during downstream analyses (e.g., in situ sequencing and detection, etc.).

### Example 2: Oligonucleotide Probes and Splints for the Stabilization and/or Compaction of a Rolling Circle Amplification Product

Using a fluorescently labeled compaction oligo (“CO”) as designed in Clausson et al., Scientific Reports 5: 12317 (2015), the entire contents of which are incorporated herein by reference, versus a fluorescent oligo containing only a single complementary domain (also referred to as hybridizing region) (“dead CO”), it was shown that the total fluorescence signal per DNB, normalized against a second fluorescent oligo hybridized to an orthogonal separate sequence domain on the DNB in a second fluorescence color (to control for intrinsic variation in DNB size, such as those arising from stochastic processes and/or sequence bias in RCA), was statistically equivalent. These results show that each CO binds only a single site on the DNB and does not efficiently form intramolecular linkages within the DNB.

A kinetics experiment was also conducted, wherein the CO was hybridized to the DNB after RCA, instead of during RCA. Hybridization reactions were conducted in increasingly dilute concentrations of CO, while holding the total moles or number of CO molecules constant, again using the two-domain CO versus dead CO design. These experiments show that as the CO becomes more dilute, the signal from the two-domain CO decreases relative to the dead CO signal until it reaches approximately 50%, which is the expected fluorescence ratio.

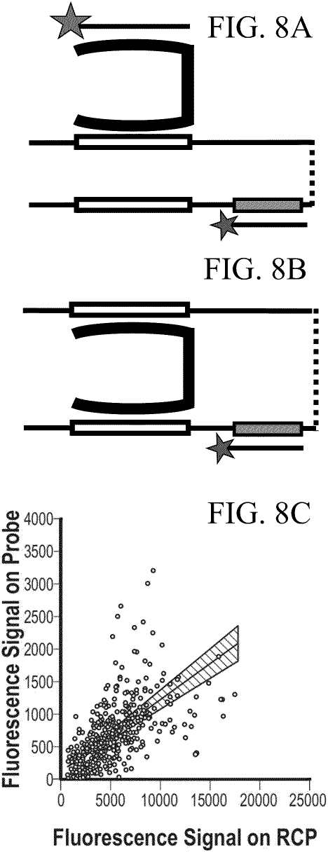

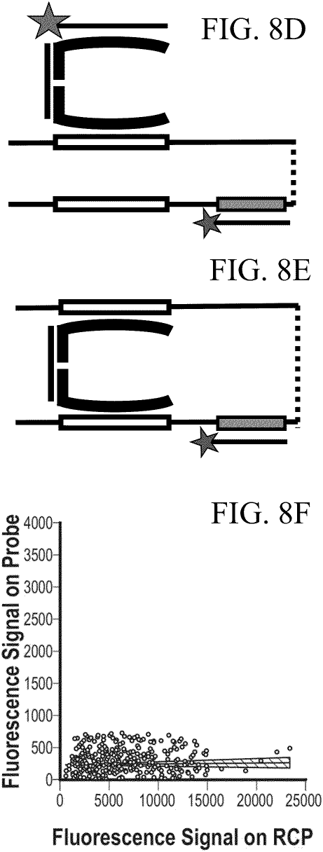

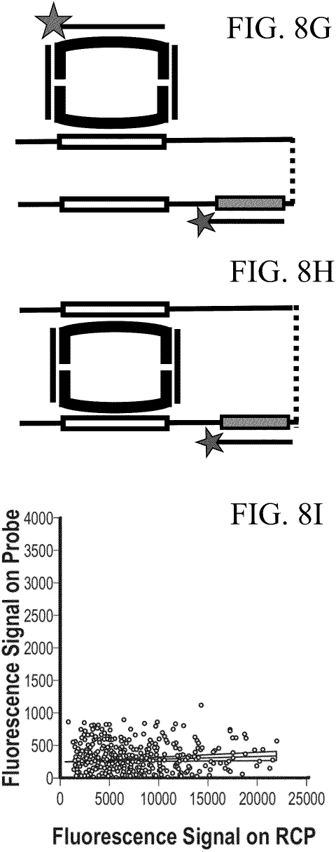

Using a first fluorescent probe hybridizing to a hybridizing region (“arm”) of the CO shown in FIG. 8A and a second fluorescent probe hybridizing to the RCP, it was also shown that increasing the reaction volume promoted a two-step binding of the CO to the RCP. Comparing signals in 400 mL reaction volume to those in 0.4 mL reaction volume, both hybridizing regions of the CO were more often hybridizing to the RCP (data not shown). In other words, at high CO concentrations, the first step is more efficient (that is, binding to the RCP by one arm of the CO is more frequent than both arms binding to the RCP), while at low CO concentrations, the first step and the second step occur at similar rates and binding to the RCP by both arms of the CO is more frequent.

In this example, a method of forming DNA nanostructures within DNBs was performed, utilizing a two-step reaction. In the first step, oligos (“rolony stabilizing components”) (RSC) containing a single DNB-hybridizing sequence domain and one or more non-DNB-hybridizing sequence domains were hybridized to the DNB. In a second step, a linkage between two or more RSCs was formed, such as by hydrogen bonding by a bridging oligonucleotide, and/or formation of chemical linkages between two or more RSCs, e.g., by enzymatic or chemical ligation.

FIG. 8A, FIG. 8B, and FIG. 8C show results of the fluorescent signal on RCP versus fluorescent signal on the free arms of the COs as described in Clausson et al., the entire contents of which are incorporated herein by reference. The steep slope of the line of best fit indicates that the fluorescent probe is able to hybridize to hybridizing regions (“free arms”) of the COs readily, indicating that the COs frequently only has one hybridizing region of its two hybridizing regions hybridized to the RCP. In contrast, results in FIG. 8D, FIG. 8E, FIG. 8F, FIG. 8G, FIG. 8H, and FIG. 81 show that fewer fluorescent detection probes hybridized to the oligonucleotide probes, because both hybridizing regions of the connected oligonucleotide probe set were more often hybridizing to the nucleic acid concatemer.

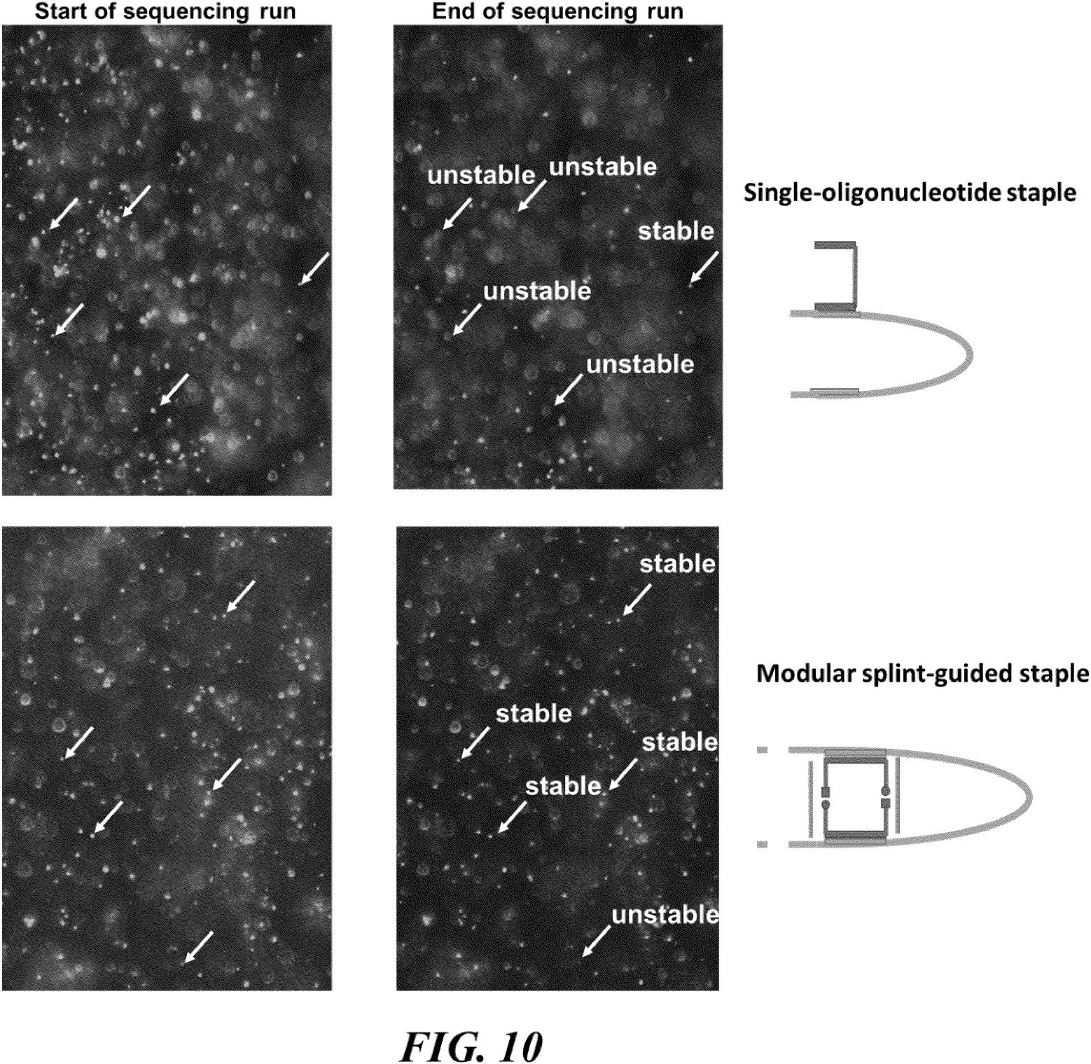

Changes in RCP integrity and stability were observed during in situ sequencing. Many RCPs appeared to lose structural integrity during multi-step sequencing runs shown in FIG. 10 (upper panel) using the COs as described in Clausson et al., the entire contents of which are incorporated herein by reference. In contrast, using exemplary oligonucleotide probes and splint disclosed herein (FIG. 10, lower panel), more RCPs appeared to maintain structural integrity during multi-step sequencing runs. These results show that modular splint-guided staples can be more efficient at achieving amplicon crosslinking and stabilization.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO. | SEQUENCE | DESCRIPTION |
|---|---|---|
| 1 | CGTTCCTACCGCCCTAATTAT | Exemplary hybridizing region of oligonucleotide probes |
| 2 | GGCTTACGCC | Exemplary overhang of oligonucleotide probes |
| 3 | CAAGAACGTA | Exemplary overhang of oligonucleotide probes |
| 4 | GGCTTACGCCCGTTCCTACCGCCCTAATTAT | Exemplary oligonucleotide probe |
| 5 | CGTTCCTACCGCCCTAATTATCAAGAACGTA | Exemplary oligonucleotide probe |
| 6 | GGCGTAAGCCTACGTTCTTG | Exemplary Splint |
| 7 | GGCTTACGCCCGTTCCTACCGCCCTAATTATCAAG AACGTA | Exemplary oligonucleotide probe |
| 8 | GGCTTACGCCCGTTCCTACCGCCCTAATTATTTTC GTTCCTACCGCCCTAATTATCAAGAACGTA | Exemplary oligonucleotide probe |
| | TTT | Exemplary Linker |
| 9 | CGTTCCTACCTTTCGTTCCTACC | Exemplary oligonucleotide probe |
| 10 | GCCCTAATTATTTTGCCCTAATTAT | Exemplary oligonucleotide probe |
| 11 | CGTTCCTACC | Exemplary hybridizing region |
| 12 | GCCCTAATTAT | Exemplary hybridizing region |
| 13 | CGTTCCTACCTCACCGCTTCCGTTCCTACC | Exemplary oligonucleotide probe |
| 14 | GCCCTAATTATGAAGCGGTGAGCCCTAATTAT | Exemplary oligonucleotide probe |
| 15 | TCACCGCTTC | Exemplary Linker |
| 16 | GAAGCGGTGA | Exemplary Linker |

1. A method for analyzing a biological sample, comprising:
(a) contacting the biological sample with one or more oligonucleotide probes, wherein:
a nucleic acid concatemer comprising multiple copies of an oligonucleotide probe recognition site is generated in the biological sample, and
each oligonucleotide probe comprises (i) a hybridizing region that hybridizes to the oligonucleotide probe recognition site or a portion thereof, and (ii) an overhang that does not hybridize to the nucleic acid concatemer; and
(b) connecting the overhangs of the one or more oligonucleotide probes hybridized to different copies of the oligonucleotide probe recognition site.

2. The method of claim 1, wherein the biological sample is in contact with the one or more oligonucleotide probes prior to or at the initiation of the generation of the nucleic acid concatemer.

3. (canceled)

4. The method of claim 1, wherein the biological sample is in contact with the one or more oligonucleotide probes after the generation of the nucleic acid concatemer.

5. (canceled)

6. The method of claim 1, wherein in the connecting step, the ends of the overhangs are hybridized to a splint wherein the splint is between about 5 and about 50 nucleotides in length.

7-13. (canceled)

14. The method of claim 1, wherein the 5′ and/or 3′ ends of the one or more oligonucleotide probes comprise click functional groups.

15. The method of claim 14, wherein the click functional groups are selected from the group consisting of:
(i) 3′-azido / 5′-alkynyl;
(ii) 3′-alkynyl / 5′-azido;
(iii) 3′-azido / 5′-cyclooctynyl;
(iv) 3′-cyclooctynyl / 5′-azido;
(v) 3′-tetrazine / 5′-dienophile;
(vi) 3′-dienophile / 5′-tetrazine;
(vii) 3′-thiol / 5′-alkynyl;
(viii) 3′-alkynyl / 5′-thiol;
(ix) 3′-cyano / 5′-1,2-amino thiol;
(x) 3′-1,2-amino thiol / 5′- cyano;
(xi) 3′-nitrone / 5′-cyclooctynyl; and
(xii) 3′-cyclooctynyl / 5′-nitrone.

16. (canceled)

17. The method of claim 1, wherein in the connecting step, the ends of an oligonucleotide probe are connected, wherein the oligonucleotide probe comprises (i) hybridizing regions that hybridize to different copies of the oligonucleotide probe recognition site and (ii) a linker between the hybridizing regions that does not hybridize to the nucleic acid concatemer, and wherein the linker is between about 0 and about 200 nucleotides in length.

18. (canceled)

19. The method of claim 1, wherein in the connecting step, an end of a first oligonucleotide probe and an end of a second oligonucleotide probe are connected, and wherein the first and second oligonucleotide probes comprise hybridizing regions that hybridize to different copies of the oligonucleotide probe recognition site.

20. The method of claim 19, wherein:
the first oligonucleotide probe comprises a 5′ azide and a 5′ sequence that hybridizes to a splint,
the second oligonucleotide probe comprises a 3′ alkyne and a 3′ sequence that hybridizes to the splint, and
upon hybridization of the first and second oligonucleotide probes to the splint, the 5′ azide and 3′ alkyne are in proximity for click reaction.

21. The method of claim 19, wherein:
the first oligonucleotide probe comprises (i) a 5′ azide and a 5′ sequence that hybridizes to a first splint, and (ii) a 3′ alkyne and a 3′ sequence that hybridizes to a second splint,
the second oligonucleotide probe comprises (i) a 3′ alkyne and a 3′ sequence that hybridizes to the first splint, and (ii) a 5′ azide and a 5′ sequence that hybridizes to the second splint, and
upon hybridization of the first and second oligonucleotide probes to the first and second splints, the 5′ azide of the first oligonucleotide and the 3′ alkyne of the second oligonucleotide are in proximity for click reaction, and the 3′ alkyne of the first oligonucleotide and the 5′ azide of the second oligonucleotide are in proximity for click reaction.

22-27. (canceled)

28. The method of claim 1, wherein the oligonucleotide probe recognition site comprises a first recognition sequence and a second recognition sequence.

29-30. (canceled)

31. The method of claim 1, wherein the nucleic acid concatemer is generated and analyzed in situ in the biological sample.

32-33. (canceled)

34. The method of claim 1, wherein the biological sample is embedded in a matrix, wherein the matrix is a hydrogel.

35-40. (canceled)

41. The method of claim 1, wherein the method comprises imaging the biological sample to detect the nucleic acid concatemer, wherein the imaging comprises fluorescent microscopy.

42-47. (canceled)

48. The method of claim 1, wherein the nucleic acid concatemer is a rolling circle amplification (RCA) product (RCP) of a circular or circularizable probe or probe set that hybridizes to a DNA or RNA molecule in the biological sample.

49. The method of claim 48, wherein the nucleic acid concatemers generated from a plurality of different mRNA and/or cDNA molecules are analyzed, a barcode sequence in a particular circular or circularizable probe or probe set uniquely corresponds to a particular mRNA or cDNA molecule.

50-52. (canceled)

53. The method of claim 1, wherein the biological sample is a non-homogenized tissue sample or a tissue section.

54-56. (canceled)

57. A method for analyzing a biological sample, comprising:
(a) contacting the biological sample with a circular probe or circularizable probe or probe set comprising a sequence that hybridizes to a target nucleic acid in the biological sample and a sequence complementary to an oligonucleotide probe recognition site,
wherein a rolling circle amplification product (RCP) is generated in the biological sample using the circular probe or a circularized probe generated from the circularizable probe or probe set as a template, wherein the RCP comprises multiple copies of the oligonucleotide probe recognition site;
(b) contacting the biological sample with a first oligonucleotide probe and a second oligonucleotide probe each comprising (i) a hybridizing region complementary to the oligonucleotide probe recognition site or a portion thereof, and (ii) an overhang that does not hybridize to the RCP, wherein the hybridizing regions of the first and second oligonucleotide probes are hybridized to different copies of the oligonucleotide probe recognition site;
(c) contacting the biological sample with a splint that hybridizes to the overhangs of the first and second oligonucleotide probes; and
(d) ligating the overhangs hybridized to the splint, thereby forming a ligated product of the first and second oligonucleotide probes hybridized to different copies of the oligonucleotide probe recognition site in the RCP.

58-59. (canceled)

60. A method for analyzing a biological sample, comprising:
(a) contacting the biological sample with a circular probe or circularizable probe or probe set comprising a sequence that hybridizes to a target nucleic acid in the biological sample and a sequence complementary to an oligonucleotide probe recognition site,
wherein a rolling circle amplification product (RCP) is generated in the biological sample using the circular probe or a circularized probe generated from the circularizable probe or probe set as a template, wherein the RCP comprises multiple copies of the oligonucleotide probe recognition site;
(b) contacting the biological sample with a first oligonucleotide probe and a second oligonucleotide probe each comprising (i) a hybridizing region complementary to the oligonucleotide probe recognition site or a portion thereof, and (ii) a 5′ overhang and a 3′ overhang that do not hybridize to the RCP, wherein the hybridizing regions of the first and second oligonucleotide probes are hybridized to different copies of the oligonucleotide probe recognition site;
(c) contacting the biological sample with a first splint that hybridizes to the 5′ overhang of the first oligonucleotide probe and the 3′ overhang of the second oligonucleotide probe, and with a second splint that hybridizes to the 3′ overhang of the first oligonucleotide probe and the 5′ overhang of the second oligonucleotide probe; and
(d) ligating the overhangs hybridized to the first splint and the overhangs hybridized to the second splint, thereby forming a ligated product of the first and second oligonucleotide probes hybridized to different copies of the oligonucleotide probe recognition site in the RCP.

61-67. (canceled)

68. The method of claim 57, further comprising crosslinking the ligated product to itself, to one or more other molecules in the biological sample, and/or to a matrix embedding the biological sample.

69-72. (canceled)
